SEQUENCE LISTING (17 sequences)

SEQ ID NO: 1 - DNA, 7524 bp, Artificial Sequence
  Synthesized lentiviral vector encoding an iduronate 2-sulfatase (I2S) polypeptide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatcatatgccagcctatggtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagtagggtgcgagagcgtcggtattaagcgggggagaattagataaatgggaaaaaattcggttaaggccagggggaaagaaacaatataaactaaaacatatagttagggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaattaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatagctctagagcgatcccgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccatctcgacggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgattcacctgacgcgtctacgccaccatgcctccaccgcggacagggaggggtctgctctggctgggactggtcctttcatccgtctgcgtggcgctgggatccgagactcaggccaattccactactgacgcgctgaacgtgctgctgattatcgtggatgatcttcgcccctcgcttggatgctacggggataagctcgtccggagcccgaacattgaccagttggcatcccactccctcctgtttcaaaacgccttcgcacaacaagccgtgtgcgctccgagcagagtgtcgttcctgaccggccggcgccctgacaccacccggttgtacgacttcaactcctattggcgcgtccacgcgggaaacttttcaaccatcccgcagtacttcaaggaaaacggttacgtgaccatgtccgtgggaaaagtgttccaccccggcatctcgtcgaatcataccgatgacagcccttactcctggtcgttccccccctatcacccgtcaagcgaaaaatacgagaacaccaagacctgtagaggtcccgacggagaactgcacgctaacctcctgtgccccgtggacgtgctggacgtgcctgaagggacccttcccgacaagcagtcaaccgagcaggccatccagctgctggaaaagatgaaaacttcggccagccccttcttcctcgccgtgggataccataagcctcatatccccttccggtatcccaaggagttccagaagctgtatccactcgagaacatcactctggccccggatccggaggtgcccgacggactgccacctgtggcctacaacccatggatggacattcggcagcgggaggatgtgcaggccctcaacatttccgtcccgtacgggccgatccctgtggacttccagcgcaagatccgacagtcctacttcgcctccgtgtcttacctggatactcaagtcgggcggctgctctccgcgctggacgatctccagcttgcaaatagcacgatcatcgccttcacctccgatcacggatgggccctgggagaacacggcgaatgggcgaagtactccaacttcgacgtggccactcacgtgccgctgatcttttacgtgccgggcagaaccgcctccctcccggaagccggagagaagctgtttccgtacctggacccgttcgactccgcgagccagctcatggagcccgggcgccagagcatggacctggtcgaactcgtgtcgctgtttcccaccctggctggcctcgccggtttgcaagtgcccccgaggtgccctgtgccgagcttccatgtggaactgtgcagggagggaaagaacctgttgaagcacttccggttccgcgacctggaggaagatccgtacttgcctggcaaccctagagaactgatcgcctactcccaataccctcggccttcggacatccctcagtggaactccgacaagccatccctgaaagacatcaagattatgggatacagcattcgcactatcgactaccgctacactgtgtgggtcggcttcaaccccgatgagttcctggccaacttctccgacattcatgctggcgaactgtacttcgtggactcagacccactccaagaccacaacatgtacaacgactcacagggaggcgatctgtttcaactcctgatgccctagtaatgacaggtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaatgtgtgtgttggttttttgtgtgtcgaaattctagcgattctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggactagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgagcttgcatgccgacattgattattgactagtccctaagaaaccattcttatcatgacattaacctataaaaataggcgtatcacgaggccctttcgtc

SEQ ID NO: 2 - DNA, 7658 bp, Artificial Sequence
  Synthesized lentiviral vector encoding an I2S polypeptide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatcatatgccagcctatggtgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgggtctctctggttagaccagatctgagcctgggagctctctggctaactagggaacccactgcttaagcctcaataaagcttgccttgagtgctcaaagtagtgtgtgcccgtctgttgtgtgactctggtaactagagatccctcagacccttttagtcagtgtggaaaatctctagcagtggcgcccgaacagggacttgaaagcgaaagtaaagccagaggagatctctcgacgcaggactcggcttgctgaagcgcgcacggcaagaggcgaggggcggcgactggtgagtacgccaaaaattttgactagcggaggctagaaggagagagtagggtgcgagagcgtcggtattaagcgggggagaattagataaatgggaaaaaattcggttaaggccagggggaaagaaacaatataaactaaaacatatagttagggcaagcagggagctagaacgattcgcagttaatcctggccttttagagacatcagaaggctgtagacaaatactgggacagctacaaccatcccttcagacaggatcagaagaacttagatcattatataatacaatagcagtcctctattgtgtgcatcaaaggatagatgtaaaagacaccaaggaagccttagataagatagaggaagagcaaaacaaaagtaagaaaaaggcacagcaagcagcagctgacacaggaaacaacagccaggtcagccaaaattaccctatagtgcagaacctccaggggcaaatggtacatcaggccatatcacctagaactttaaattaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaacacatggaaaagattagtaaaacaccatagctctagagcgatcccgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacgcgtctacgccaccatgcctccaccgcggacagggaggggtctgctctggctgggactggtcctttcatccgtctgcgtggcgctgggatccgagactcaggccaattccactactgacgcgctgaacgtgctgctgattatcgtggatgatcttcgcccctcgcttggatgctacggggataagctcgtccggagcccgaacattgaccagttggcatcccactccctcctgtttcaaaacgccttcgcacaacaagccgtgtgcgctccgagcagagtgtcgttcctgaccggccggcgccctgacaccacccggttgtacgacttcaactcctattggcgcgtccacgcgggaaacttttcaaccatcccgcagtacttcaaggaaaacggttacgtgaccatgtccgtgggaaaagtgttccaccccggcatctcgtcgaatcataccgatgacagcccttactcctggtcgttccccccctatcacccgtcaagcgaaaaatacgagaacaccaagacctgtagaggtcccgacggagaactgcacgctaacctcctgtgccccgtggacgtgctggacgtgcctgaagggacccttcccgacaagcagtcaaccgagcaggccatccagctgctggaaaagatgaaaacttcggccagccccttcttcctcgccgtgggataccataagcctcatatccccttccggtatcccaaggagttccagaagctgtatccactcgagaacatcactctggccccggatccggaggtgcccgacggactgccacctgtggcctacaacccatggatggacattcggcagcgggaggatgtgcaggccctcaacatttccgtcccgtacgggccgatccctgtggacttccagcgcaagatccgacagtcctacttcgcctccgtgtcttacctggatactcaagtcgggcggctgctctccgcgctggacgatctccagcttgcaaatagcacgatcatcgccttcacctccgatcacggatgggccctgggagaacacggcgaatgggcgaagtactccaacttcgacgtggccactcacgtgccgctgatcttttacgtgccgggcagaaccgcctccctcccggaagccggagagaagctgtttccgtacctggacccgttcgactccgcgagccagctcatggagcccgggcgccagagcatggacctggtcgaactcgtgtcgctgtttcccaccctggctggcctcgccggtttgcaagtgcccccgaggtgccctgtgccgagcttccatgtggaactgtgcagggagggaaagaacctgttgaagcacttccggttccgcgacctggaggaagatccgtacttgcctggcaaccctagagaactgatcgcctactcccaataccctcggccttcggacatccctcagtggaactccgacaagccatccctgaaagacatcaagattatgggatacagcattcgcactatcgactaccgctacactgtgtgggtcggcttcaaccccgatgagttcctggccaacttctccgacattcatgctggcgaactgtacttcgtggactcagacccactccaagaccacaacatgtacaacgactcacagggaggcgatctgtttcaactcctgatgccctagtaatgacaggtacctttaagaccaatgacttacaaggcagctgtagatcttagccactttttaaaagaaaaggggggactggaagggctaattcactcccaaagaagacaagatctgctttttgcctgtactgggtctctctggttagaccagatctgagcctgggagctctctggctaactagggaacccactgcttaagcctcaataaagcttgccttgagtgcttcaatgtgtgtgttggttttttgtgtgtcgaaattctagcgattctagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggactagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgagcttgcatgccgacattgattattgactagtccctaagaaaccattcttatcatgacattaacctataaaaataggcgtatcacgaggccctttcgtc

SEQ ID NO: 3 - protein, 3 aa, Artificial Sequence
  Exemplary linker sequence
GGG

SEQ ID NO: 4 - protein, 5 aa, Artificial Sequence
  Exemplary linker sequence
DGGGS

SEQ ID NO: 5 - protein, 5 aa, Artificial Sequence
  Exemplary linker sequence
TGEKP

SEQ ID NO: 6 - protein, 4 aa, Artificial Sequence
  Exemplary linker sequence
GGRR

SEQ ID NO: 7 - protein, 5 aa, Artificial Sequence
  Exemplary linker sequence
GGGGS

SEQ ID NO: 8 - protein, 14 aa, Artificial Sequence
  Exemplary linker sequence
EGKSSGSGSESKVD

SEQ ID NO: 9 - protein, 18 aa, Artificial Sequence
  Exemplary linker sequence
KESGSVSSEQLAQFRSLD

SEQ ID NO: 10 - protein, 8 aa, Artificial Sequence
  Exemplary linker sequence
GGRRGGGS

SEQ ID NO: 11 - protein, 9 aa, Artificial Sequence
  Exemplary linker sequence
LRQRDGERP

SEQ ID NO: 12 - protein, 12 aa, Artificial Sequence
  Exemplary linker sequence
LRQKDGGGSERP

SEQ ID NO: 13 - protein, 16 aa, Artificial Sequence
  Exemplary linker sequence
LRQKDGGGSGGGSERP

SEQ ID NO: 14 - protein, 7 aa, Artificial Sequence
  Cleavage sequence by TEV protease
  misc_feature (2)..(3): Xaa is any amino acid
  misc_feature (5)..(5): Xaa is any amino acid
  MISC_FEATURE (7)..(7): Xaa = Gly or Ser
EXXYXQX

SEQ ID NO: 15 - protein, 7 aa, Artificial Sequence
  Cleavage sequence by TEV protease
ENLYFQG

SEQ ID NO: 16 - protein, 7 aa, Artificial Sequence
  Cleavage sequence by TEV protease
ENLYFQS

SEQ ID NO: 17 - DNA, 10 bp, Artificial Sequence
  Consensus Kozak sequence
gccrccatgg

(19) United States
(12) Patent Application Publication
(10) Pub. No.: US 2020/0071721 A1
(43) Pub. Date: Mar. 5, 2020

(54) GENE THERAPY FOR MUCOPOLYSACCHARIDOSIS, TYPE II

(71) Applicant: bluebird bio, Inc., Cambridge, MA (US)

(72) Inventors: Kendrick A. GOSS, Lexington, MA (US); Geoffrey B. PARSONS, Jamaica Plain, MA (US)

(73) Assignee: bluebird bio, Inc., Cambridge, MA (US)

(21) Appl. No.: 16/466,123

(22) PCT Filed: Dec. 6, 2017
(86) PCT No.: PCT/US17/64940

§ 371 Date: Jun. 3, 2019

Related U.S. Application Data
(60) Provisional application No. 62/430,819, filed on Dec. 6, 2016.

(51) Int. Cl. C12N 15/86; C12N 9/16; A61K 48/00; A61P 3/00; C12N 5/0789

(52) U.S. Cl. CPC C12N 15/86; C12N 9/16; A61K 48/005; A61K 38/00; A61K 48/0083; C12N 5/0647; C12Y 301/06013; A61P 3/00

(57) ABSTRACT

The invention provides compositions and methods for treating Hunter Syndrome.

### CROSS REFERENCE TO RELATED APPLICATIONS

This application claims the benefit under 35 U.S.C. § 119(e) of U.S. Provisional Application No. 62/430,819, filed Dec. 6, 2016, which is incorporated by reference herein in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is BLBD 082_01_WO_ST25.txt. The text file is 24 KB, was created on Dec. 6, 2017, and is being submitted electronically via EFS-Web, concurrent with the filing of the specification.

### BACKGROUND

### Technical Field

The present invention relates to gene therapy. More particularly, the invention relates to gene therapy compositions and methods of using the same to treat mucopolysaccharidosis, type II (MPS II), also known as Hunter syndrome.

### Description of the Related Art

Mucopolysaccharidoses (MPS) are a class of serious genetic disorders known as lysosomal storage diseases. MPS interferes with the body's ability to continuously break down and recycle specific mucopolysaccharides.

Mucopolysaccharidosis Type II (MPS II) or Hunter syndrome is an X-linked recessive mucopolysaccharide disease that affects an estimated 1 out of every 100,000 to 150,000 males in the United States alone; on rare occasion heterozygous females manifest the disease.

Children with MPS II have a defective copy of an iduronate 2-sulfatase gene (IDS) that encodes the enzyme iduronate 2-sulfatase (I2S). I2S is responsible for breaking down large sugar molecules known as glycosaminoglycans (GAGs) or mucopolysaccharides. Loss of I2S function allows undigested dermatan sulfate and heparan sulphate and other harmful substances to build up in cells throughout the body, resulting in the eventual damage or destruction to almost every system of the body.

Age of onset, disease severity, and rate of progression Hunter Syndrome vary significantly among affected males. Hunter syndrome symptoms usually manifest between 2 and 4 years of age. It is difficult to detect Hunter syndrome, because most symptoms mimic common childhood sicknesses. Most cases of Hunter syndrome are diagnosed from signs of developmental delay as children begin school.

In those with early progressive disease, CNS involvement (manifest primarily by progressive cognitive deterioration), progressive airway disease, and cardiac disease. In those with slowly progressive disease, the CNS is not (or is minimally) affected, although the effect of GAG accumulation on other organ systems may be early progressive to the same degree as in those who have progressive cognitive decline. Survival into the early adult years with normal intelligence is common in the slowly progressing form of the disease. Additional findings in both forms of Hunter Syndrome include: short stature; macrocephaly with or without communicating hydrocephalus; macroglossia; hoarse voice; conductive and sensorineural hearing loss; hepato-splenomegaly; dysostosis multiplex; spinal stenosis; and carpal tunnel syndrome.

Though treatment may improve the length and quality of life for children with Hunter Syndrome, there is no cure for Hunter syndrome and those affected with the severe form often die before reaching their mid-teens from heart disease, airway obstruction, or severe neurological damage.

### BRIEF SUMMARY

The invention generally relates, in part, to gene therapy compositions and methods for the treatment, prevention, or amelioration of at least one symptom of Mucopolysaccharidosis Type II (MPS II) or Hunter syndrome.

In various embodiments, a polynucleotide is provided comprising: a left (5′) lentiviral LTR; a Psi (w) packaging signal; a retroviral export element; a central polypurine tract/DNA flap (cPPT/FLAP); a promoter operably linked to a polynucleotide encoding iduronate 2-sulfatase (I2S) polypeptide; and a right (3′) lentiviral LTR.

In particular embodiments, the lentivirus is selected from the group consisting of: HIV (human immunodeficiency virus; including HIV type 1, and HIV type 2); visna-maedi virus (VMV) virus; caprine arthritis-encephalitis virus (CAEV); equine infectious anemia virus (EIAV); feline immunodeficiency virus (Hy); bovine immune deficiency virus (BIV); and simian immunodeficiency virus (SIV).

In certain embodiments, the lentivirus is HIV-1 or HIV-2.

In some embodiments, the lentivirus is HIV-1.

In additional embodiments, the promoter of the 5′ LTR is replaced with a heterologous promoter selected from the group consisting of: a cytomegalovirus (CMV) promoter, a Rous Sarcoma Virus (RSV) promoter, and a Simian Virus 40 (SV40) promoter.

In further embodiments, the 3′ LTR comprises one or more modifications.

In some embodiments, the 3′ LTR comprises one or more deletions that prevent viral transcription beyond the first round of viral replication.

In particular embodiments, the 3′ LTR comprises a deletion of the TATA box and Sp1 and NF-κB transcription factor binding sites in the U3 region of the 3′ LTR.

In some embodiments, the 3′ LTR is a self-inactivating (SIN) LTR.

In certain embodiments, the promoter operably linked to a polynucleotide encoding an I2S polypeptide is selected from the group consisting of: an integrin subunit alpha M (ITGAM; CD11b) promoter, a CD68 promoter, a C-X3-C motif chemokine receptor 1 (CX3CR1) promoter, an ionized calcium binding adaptor molecule 1 (IBA1) promoter, a transmembrane protein 119 (TMEM119) promoter, a spalt like transcription factor 1 (SALL1) promoter, an adhesion G protein-coupled receptor E1 (F4/80) promoter, a myeloproliferative sarcoma virus enhancer, negative control region deleted, d1587rev primer-binding site substituted (MND) promoter and transcriptionally active fragments thereof.

In certain embodiments, the promoter operably linked to a polynucleotide encoding an I2S polypeptide comprises a myeloproliferative sarcoma virus enhancer, negative control region deleted, d1587rev primer-binding site substituted (MND) promoter or transcriptionally active fragment thereof.

In additional embodiments, the promoter operably linked to a polynucleotide encoding an I2S polypeptide comprises an elongation factor 1 alpha (EF1α) promoter or transcriptionally active fragment thereof.

In particular embodiments, the promoter operably linked to a polynucleotide encoding an I2S polypeptide is a short EF1α promoter.

In some embodiments, the promoter operably linked to a polynucleotide encoding an I2S polypeptide is a long EF1α promoter.

In further embodiments, the polynucleotide encoding the I2S polypeptide is a cDNA.

In particular embodiments, the polynucleotide encoding the I2S polypeptide is codon optimized for expression.

In particular embodiments, a polynucleotide is provided, comprising: a left (5′) HIV-1 LTR; a Psi (w) packaging signal; an RRE retroviral export element; a cPPT/FLAP; an MND promoter or EF1α promoter operably linked to a polynucleotide encoding an 12S polypeptide; and a right (3′) HIV-1 LTR.

In particular embodiments, a polynucleotide is provided, comprising: a left (5′) CMV promoter/HIV-1 chimeric LTR; a Psi (ψ) packaging signal; an RRE retroviral export element; a cPPT/FLAP; an MND promoter or EF1α promoter operably linked to a polynucleotide encoding an I2S polypeptide; and a right (3′) SIN HIV-1 LTR.

In particular embodiments, the polynucleotide further comprise a bovine growth hormone polyadenylation signal or a rabbit β-globin polyadenylation signal.

In various embodiments, a mammalian cell transduced with a lentiviral vector is provided, comprising a polynucleotide contemplated herein.

In some embodiments, the cell is a hematopoietic cell.

In certain embodiments, the cell is a CD34+ cell.

In particular embodiments, the cell is a stem cell or progenitor cell.

In various embodiments, a producer cell comprising: a first polynucleotide encoding gag, a second polynucleotide encoding pol, a third polynucleotide encoding env, and a polynucleotide contemplated herein.

In various particular embodiments, a lentiviral vector produced by the producer cell contemplated herein is provided.

In various certain embodiments, a composition comprising a lentiviral vector comprising a polynucleotide or a mammalian cell contemplated herein is provided.

In various further embodiments, a pharmaceutical composition comprising a pharmaceutically acceptable carrier and a lentiviral vector comprising a polynucleotide or a mammalian cell contemplated herein is provided.

In various additional embodiments, a method of treating Hunter Syndrome, comprising administering to a subject a lentiviral vector comprising a polynucleotide, a cell transduced with a lentiviral vector comprising a polynucleotide, or a mammalian cell contemplated herein is provided.

In various some embodiments, a method of treating Hunter Syndrome, comprising administering to a subject a pharmaceutical composition contemplated herein is provided.

In various particular embodiments, a method of decreasing at least one symptom associated with Hunter Syndrome in a subject comprising administering to a subject a lentiviral vector comprising a polynucleotide, a cell transduced with a lentiviral vector comprising a polynucleotide, or a mammalian cell contemplated herein is provided.

In various embodiments, a method of decreasing at least one symptom associated with Hunter Syndrome in a subject is provided, comprising administering to a subject a pharmaceutical composition contemplated herein.

In some embodiments, at least one symptom is selected from the group consisting of: build up of GAGs, thickening of organ and tissues, difficulty breathing, difficulty swallowing, joint stiffness, cognitive function decline, and motor function decline.

### BRIEF DESCRIPTION OF SEVERAL VIEWS OF THE DRAWINGS

FIG. 1 shows exemplary architectures of lentiviral vectors encoding 12S.

FIG. 2 shows the data from a representative experiment assaying 12S enzymatic activity in wild type control cells, 12S^{-/-} cells, and I2S^{-/-} cells transduced with the lentiviral vectors encoding IDUA (pMND-I2S and pEF1α-I2S).

FIG. 3 shows that human CD34⁺ cells transduced with LVV comprising an MND or EF1α promoter linked to a polynucleotide encoding I2S exhibited similar growth kinetics compared to mock transduced cells.

FIG. 4 shows the VCN of human CD34⁺ cells transduced with LVV comprising an MND or EF1α promoter linked to a polynucleotide encoding I2S and cultured with cytokines for 7 or 14 days.

FIG. 5 shows individual colony VCNs of human CD34⁺ cells transduced with LVV comprising an MND or EF1α promoter linked to a polynucleotide encoding I2S at day 12 in methylcellulose culture.

FIG. 6 shows IDUA activity in cell pellets from human CD34⁺ cells transduced with LVV comprising an MND or EF1α promoter linked to a polynucleotide encoding IDUA I and cultured with cytokines for 7 days.

### BRIEF DESCRIPTION OF THE SEQUENCE IDENTIFIERS

SEQ ID NO: 1 sets forth the sequence of an exemplary lentiviral vector encoding an iduronate 2-sulfatase (I2S) polypeptide.

SEQ ID NO: 2 sets forth the sequence of an exemplary lentiviral vector encoding an I2S polypeptide.

SEQ ID NOs: 3-13 set forth the amino acid sequences of various linkers.

SEQ ID NOs: 14-16 set forth the amino acid sequences of protease cleavage sites and self-cleaving polypeptide cleavage sites.

### DETAILED DESCRIPTION

### A. Overview

The invention generally relates, in part, to improved gene therapy compositions and methods for treating, preventing, or ameliorating at least one symptom of MPSII or Hunter Syndrome.

Hunter Syndrome is an inherited disorder for which there is no clinically approved curative treatment and for which palliative care is the only option. Hunter Syndrome is a lysosomal storage disease, marked by a buildup of substances called mucopolysaccharides or glycosaminoglycans (GAGs) in membrane bound organelles called lysosomes. Lysosomes are compartments in the cell that normally digest and recycle different types of molecules. The accumulation of GAGs in lysosomes occurs in cells throughout the body, and leads to tissue and organ damage and dysfunction and often death before age 20. The progressive death of cells, especially in the central nervous system, leads decline in motor function and cognitive ability in children with Hunter Syndrome.

In various embodiments, a gene therapy vector encoding an iduronate 2-sulfatase (I2S) polypeptide is contemplated. The gene therapy preferentially includes a promoter operably linked to the polynucleotide encoding the I2S polypeptide. The gene therapy vector may be a viral vector, including but not limited to a gammaretroviral vector, a lentiviral vector, an adeno-associated viral (AAV) vector, an adenoviral vector, or a herpes virus vector.

Cells transduced with the gene therapy vectors contemplated herein are also provided in various embodiments. In some preferred embodiments, the transduced cells are hematopoietic cells, including, but not limited to CD34⁺ cells.

In various other embodiments, gene therapy compositions contemplated herein are preferably administered to a subject that has a subject that has been diagnosed with or that has Hunter Syndrome.

In various other embodiments, gene therapy compositions contemplated herein are preferably administered to a subject that has a subject that has one or more mutations in an I2S gene.

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

By “enhance” or “promote,” or “increase” or “expand” refers generally to the ability of the compositions and/or methods contemplated herein to elicit, cause, or produce higher physiological response compared to vehicle or a control molecule/composition. An “increased” or “enhanced” amount is typically a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount of a control.

By “decrease” or “lower,” or “lessen,” or “reduce,” or “abate” refers generally to compositions or methods that result in a decreased physiological response compared to the response of a vehicle or control composition or method. A “decrease” or “reduced” amount of transduced cells is typically a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount of a control.

By “maintain,” or “preserve,” or “maintenance,” or “no change,” or “no substantial change,” or “no substantial decrease” refers generally to a physiological response that is comparable to a response caused by either vehicle, a control molecule/composition, or the response in a particular cell. A comparable response is one that is not significantly different or measurable different from the reference response.

In the following description, certain specific details are set forth in order to provide a thorough understanding of various illustrative embodiments of the invention contemplated herein. However, one skilled in the art will understand that particular illustrative embodiments may be practiced without these details. In addition, it should be understood that the individual vectors, or groups of vectors, derived from the various combinations of the structures and substituents described herein, are disclosed by the present application to the same extent as if each vector or group of vectors was set forth individually. Thus, selection of particular vector structures or particular substituents is within the scope of the present disclosure.

### C. Polypeptides

“Polypeptide,” “polypeptide fragment,” “peptide” and “protein” are used interchangeably, unless specified to the contrary, and according to conventional meaning, i.e., as a sequence of amino acids. In one embodiment, a “polypeptide” includes fusion polypeptides and other variants. Polypeptides can be prepared using any of a variety of well-known recombinant and/or synthetic techniques. Polypeptides are not limited to a specific length, e.g., they may comprise a full length protein sequence, a fragment of a full length protein, or a fusion protein, and may include post-translational modifications of the polypeptide, for example, glycosylations, acetylations, phosphorylations and the like, as well as other modifications known in the art, both naturally occurring and non-naturally occurring.

In various embodiments, polypeptides are contemplated herein, including, but not limited to, I2S polypeptides.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances.

Polypeptides include “polypeptide variants.” Polypeptide variants may differ from a naturally occurring polypeptide in one or more amino acid substitutions, deletions, additions and/or insertions. Such variants may be naturally occurring or may be synthetically generated, for example, by modifying one or more amino acids of the above polypeptide sequences. For example, in particular embodiments, it may be desirable to modulate the biological properties of a polypeptide by introducing one or more substitutions, deletions, additions and/or insertions into the polypeptide. In particular embodiments, polypeptides include polypeptide variants having at least about 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity to any of the reference sequences contemplated herein, typically where the variant maintains at least one biological activity of the reference sequence.

Polypeptides variants include biologically active “polypeptide fragments.” As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. Polypeptide fragments refer to a polypeptide, which can be monomeric or multimeric that has an amino-terminal deletion, a carboxyl-terminal deletion, and/or an internal deletion or substitution of one or more amino acids of a naturally-occurring or recombinantly-produced polypeptide. In certain embodiments, a polypeptide fragment can comprise an amino acid chain at least 5 to about 1700 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more amino acids long.

Illustrative examples of polypeptide fragments include catalytic domains and the like.

As noted above, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments, polypeptides include polypeptides having at least about and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence.

Guidance in determining which amino acid residues can be substituted, inserted, or deleted without abolishing biological activity can be found using computer programs well known in the art, such as DNASTAR, DNA Strider, Geneious, Mac Vector, or Vector NTI software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224).

In making such changes, the hydropathic index of amino acids may be considered. The importance of the hydropathic amino acid index in conferring interactive biologic function on a protein is generally understood in the art (Kyte and Doolittle, 1982, incorporated herein by reference). Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cysteine (+2.5); methionine (+1.9); alanine (+1.8); glycine (0.4); threonine (0.7); serine (0.8); tryptophan (0.9); tyrosine (1.3); proline (1.6); histidine (3.2); glutamate (3.5); glutamine (3.5); aspartate (3.5); asparagine (3.5); lysine (3.9); and arginine (4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (-0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

Polypeptides contemplated in particular embodiments include fusion polypeptides. In particular embodiments, fusion polypeptides and polynucleotides encoding fusion polypeptides are provided. Fusion polypeptides and fusion proteins refer to a polypeptide having at least two, three, four, five, six, seven, eight, nine, or ten polypeptide segments.

In another embodiment, two or more polypeptides can be expressed as a fusion protein that comprises one or more self-cleaving polypeptide sequences as disclosed elsewhere herein.

Fusion polypeptides can comprise one or more polypeptide domains or segments including, but are not limited to signal peptides, cell permeable peptide domains (CPP), DNA binding domains, nuclease domains, chromatin remodeling domains, histone modifying domains, epigenetic modifying domains, exodomains, extracellular ligand binding domains, antigen binding domains, transmembrane domains, intracellular signaling domains, multimerization domains, epitope tags (e.g., maltose binding protein (“MBP”), glutathione S transferase (GST), HIS6, MYC, FLAG, V5, VSV-G, and HA), polypeptide linkers, and polypeptide cleavage signals. Fusion polypeptides are typically linked C-terminus to N-terminus, although they can also be linked C-terminus to C-terminus, N-terminus to N-terminus, or N-terminus to C-terminus. In particular embodiments, the polypeptides of the fusion protein can be in any order. Fusion polypeptides or fusion proteins can also include conservatively modified variants, polymorphic variants, alleles, mutants, subsequences, and interspecies homologs, so long as the desired activity of the fusion polypeptide is preserved. Fusion polypeptides may be produced by chemical synthetic methods or by chemical linkage between the two moieties or may generally be prepared using other standard techniques. Ligated DNA sequences comprising the fusion polypeptide are operably linked to suitable transcriptional or translational control elements as disclosed elsewhere herein.

Fusion polypeptides may optionally comprises a linker that can be used to link the one or more polypeptides or domains within a polypeptide. A peptide linker sequence may be employed to separate any two or more polypeptide components by a distance sufficient to ensure that each polypeptide folds into its appropriate secondary and tertiary structures so as to allow the polypeptide domains to exert their desired functions.

Exemplary linkers include, but are not limited to the following amino acid sequences: glycine polymers (G)n; glycine-serine polymers (G1-5S1-5)n, where n is an integer of at least one, two, three, four, or five; glycine-alanine polymers; alanine-serine polymers; GGG (SEQ ID NO: 3); DGGGS (SEQ ID NO: 4); TGEKP (SEQ ID NO: 5) (see e.g., Liu et al., PNAS 5525-5530 (1997)); GGRR (SEQ ID NO: 6) (Pomerantz et al. 1995, supra); (GGGGS)n wherein n=1, 2, 3, 4 or 5 (SEQ ID NO: 7) (Kim et al., PNAS 93, 1156-1160 (1996.); EGKSSGSGSESKVD (SEQ ID NO: 8) (Chaudhary et al., 1990, Proc. Natl. Acad. Sci. U.S.A. 87:1066-1070); KESGSVSSEQLAQFRSLD (SEQ ID NO: 9) (Bird et al., 1988, Science 242:423-426), GGRRGGGS (SEQ ID NO: 10); LRQRDGERP (SEQ ID NO: 11); LRQKDGGGSERP (SEQ ID NO: 12); LRQKD(GGGS)₂ERP (SEQ ID NO: 13). Alternatively, flexible linkers can be rationally designed using a computer program capable of modeling both DNA-binding sites and the peptides themselves (Desjarlais & Berg, PNAS 90:2256-2260 (1993)) or by phage display methods.

Fusion polypeptides may further comprise a polypeptide cleavage signal between each of the polypeptide domains described herein or between an endogenous open reading frame and a polypeptide encoded by a donor repair template. In addition, a polypeptide cleavage site can be put into any linker peptide sequence. Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus Ma proteases (e.g., tobacco etch virus protease), potyvirus HC proteases, potyvirus P1 (P35) proteases, byovirus Ma proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 14), for example, ENLYFQG (SEQ ID NO: 15) and ENLYFQS (SEQ ID NO: 16), wherein X represents any amino acid (cleavage by TEV occurs between Q and G or Q and S).

In certain embodiments, the self-cleaving polypeptide site comprises a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

In various embodiments, the expression or stability of polypeptides or fusion polypeptides contemplated herein is regulated by one or more protein destabilization sequences or protein degradation sequences (degrons). Several strategies to destabilize proteins to enforce their rapid proteasomal turnover are contemplated herein.

Illustrative examples of protein destabilization sequences include, but are not limited to: the destabilization box (D box), a nine amino acid is present in cell cycle-dependent proteins that must undergo rapid and complete ubiquitin-mediated proteolysis to achieve cycling within the cell cycle (see e.g., Yamano et al. 1998. Embo J 17:5670-8); the KEN box, an APC recognition signal targeted by Cdh1 (see e.g., Pfleger et al. 2000. Genes Dev 14:655-65); the O box, a motif present in origin recognition complex protein 1 (ORC1), which is degraded at the end of M phase and throughout much of G1 by anaphase-promoting complexes (APC) activated by Fzr/Cdh1 (see e.g., Araki et al. 2005. Genes Dev 19(20):2458-2465); the A-box, a motif present in Aurora-A, which is degraded during mitotic exit by Cdh1 (see e.g., Littlepage et al. 2002. Genes Dev 16:2274-2285); PEST domains, motifs enriched in proline (P), glutamic acid (E), serine (S) and threonine (T) residues and that target proteins for rapid proteasomal destruction (Rechsteiner et al. 1996. Trens Biochem Sci. 21(7):267-271); N-end rule motifs, N-degron motifs, and ubiquitin-fusion degradation (UFD) motifs, which are rapidly processed for proteasomal destruction (see e.g., Dantuma et al. 2000. Nat Biotechnol 18:538-4).

Further illustrative examples of degrons suitable for use in particular embodiments include, but are not limited to, ligand controllable degrons and temperature regulatable degrons. Non-limiting examples of ligand controllable degrons include those stabilized by Shield 1 (see e.g., Bonger et al. 2011. Nat Chem Viol. 7(8):531-537), destabilized by auxin (see e.g., Nishimura et al. 2009. Nat Methods 6(12):917-922), and stabilized by trimethoprim (see e.g., Iwamoto et al., 2010. Chem Biol. 17(9):981-8).

Non-limiting examples of temperature regulatable degrons include, but are not limited to DHFRTS degrons (see e.g., Dohmen et al., 1994. Science 263(5151):1273-1276).

In particular embodiments, a polypeptide contemplated herein comprises one or more degradation sequences selected from the group consisting of: a D box, an O box, an A box, a KEN motif, a PEST motifs, Cyclin A and UFD domain/substrates, ligand controllable degrons, and temperature regulatable degrons.

### D. Polynucleotides

As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, short interfering RNA (siRNA), short hairpin RNA (shRNA), microRNA (miRNA), ribozymes, synthetic RNA, genomic RNA (gRNA), plus strand RNA (RNA(+)), minus strand RNA (RNA(−)), tracrRNA, crRNA, single guide RNA (sgRNA), synthetic RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference sequence.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid.

Illustrative examples of polynucleotides include, but are not limited to polynucleotides sequences set forth in SEQ ID NOs: 1-2.

In various illustrative embodiments, polynucleotides contemplated herein include, but are not limited to polynucleotides comprising expression vectors, viral vectors, transfer plasmids, expression cassettes and polynucleotides encoding an iduronate 2-sulfatase (I2S) polypeptide.

The iduronate 2-sulfatase (IDS) gene encodes I2S (also referred to as MPS II and SIDS), a member of the sulfatase family of proteins. Typically, the human I2S protein is produced as a precursor form. The precursor form of human I2S contains a signal peptide (amino acid residues 1-25 of the full length precursor), a pro-peptide (amino acid residues 26-33 of the full length precursor), and a chain (residues 34-550 of the full length precursor) that may be further processed into the 42 kDa chain (residues 34-455 of the full length precursor) and the 14 kDa chain (residues 446-550 of the full length precursor). Typically, the precursor form is also referred to as full-length precursor or full-length 12S protein, which contains 550 amino acids. This enzyme is involved in the lysosomal degradation of heparan sulfate and dermatan sulfate. Mutations in this gene are associated with the X-linked lysosomal storage disease mucopolysaccharidosis type II, also known as Hunter syndrome. Alternative splicing results in multiple transcript variants, at least one of which encodes a preproprotein that is proteolytically processed.

As used herein, the terms “polynucleotide variant” and “variant” and the like refer to polynucleotides displaying substantial sequence identity with a reference polynucleotide sequence or polynucleotides that hybridize with a reference sequence under stringent conditions. These terms also encompass polynucleotides that are distinguished from a reference polynucleotide by the addition, deletion, substitution, or modification of at least one nucleotide. Accordingly, the terms “polynucleotide variant” and “variant” include polynucleotides in which one or more nucleotides have been added or deleted, or modified, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide.

The recitations “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 70%, 75%, 80%, 85%, 90%, 95%, 96%, 97%, 98%, 99% or 100% sequence identity to any of the reference sequences described herein, typically where the polypeptide variant maintains at least one biological activity of the reference polypeptide.

An “isolated polynucleotide,” as used herein, refers to a polynucleotide that has been purified from the sequences which flank it in a naturally-occurring state, e.g., a DNA fragment that has been removed from the sequences that are normally adjacent to the fragment. In particular embodiments, an “isolated polynucleotide” refers to a complementary DNA (cDNA), a recombinant polynucleotide, a synthetic polynucleotide, or other polynucleotide that does not exist in nature and that has been made by the hand of man.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the pre-messenger (pre-mRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ AGT C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise one, two, three, four, five or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment, the nucleic acid cassette contains the sequence of a therapeutic gene used to treat, prevent, or ameliorate a genetic disorder. The cassette can be removed and inserted into a plasmid or viral vector as a single unit.

As used herein, the term “polynucleotide(s)-of-interest” refers to one or more polynucleotides, e.g., a polynucleotide encoding a polypeptide (i.e., a polypeptide-of-interest), inserted into an expression vector that is desired to be expressed. In preferred embodiments, vectors and/or plasmids of the present invention comprise one or more polynucleotides-of-interest, e.g., a polynucleotide encoding an I2S polypeptide. In certain embodiments, a polynucleotide-of-interest encodes a polypeptide that provides a therapeutic effect in the treatment, prevention, or amelioration of a neuronal ceroid lipofuscinoses, which may be referred to as a “therapeutic polypeptide,” e.g., a polynucleotide encoding an I2S polypeptide.

In a certain embodiment, a polynucleotide-of-interest comprises an inhibitory polynucleotide including, but not limited to, a crRNA, a tracrRNA, a single guide RNA (sgRNA), an siRNA, an miRNA, an shRNA, a ribozyme or another inhibitory RNA.

Polynucleotides, regardless of the length of the coding sequence itself, may be combined with other DNA sequences, such as promoters and/or enhancers, untranslated regions (UTRs), Kozak sequences, polyadenylation signals, additional restriction enzyme sites, multiple cloning sites, internal ribosomal entry sites (IRES), recombinase recognition sites (e.g., LoxP, FRT, and Att sites), termination codons, transcriptional termination signals, post-transcription response elements, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed, with the total length preferably being limited by the ease of preparation and use in the intended recombinant DNA protocol.

Polynucleotides can be prepared, manipulated, expressed and/or delivered using any of a variety of well-established techniques known and available in the art. In order to express a desired polypeptide, a nucleotide sequence encoding the polypeptide, can be inserted into appropriate vector.

Illustrative examples of vectors include, but are not limited to plasmid, autonomously replicating sequences, and transposable elements, e.g., Sleeping Beauty, PiggyBac.

Additional illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to pClneo vectors (Promega) for expression in mammalian cells; pLenti4N5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

“Expression control sequences,” “control elements,” or “regulatory sequences” present in an expression vector are those non-translated regions of the vector—origin of replication, selection cassettes, promoters, enhancers, translation initiation signals (Shine Dalgarno sequence or Kozak sequence) introns, post-transcriptional regulatory elements, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, a polynucleotide is a vector, including but not limited to expression vectors and viral vectors, and includes exogenous, endogenous, or heterologous control sequences such as promoters and/or enhancers. An “endogenous” control sequence is one which is naturally linked to a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated. A “synthetic” control sequence may comprise elements of one more endogenous and/or exogenous sequences, and/or sequences determined in vitro or in silico that provide optimal promoter and/or enhancer activity for the particular gene therapy.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, a short elongation factor 1-alpha (EF1α-short) promoter, a long elongation factor 1-alpha (EF1a-long) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin ((3-KIN), the human ROSA 26 locus Orions et al., Nature Biotechnology 25, 1477-1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) promoter (Challita et al., J Virol. 69(2):748-55 (1995)).

In a particular embodiment, it may be desirable to use a cell, cell type, cell lineage or tissue specific expression control sequence to achieve cell type specific, lineage specific, or tissue specific expression of a desired polynucleotide sequence (e.g., to express a particular nucleic acid encoding a polypeptide in only a subset of cell types, cell lineages, or tissues or during specific stages of development).

Illustrative examples of tissue specific promoters include, but are not limited to: an B29 promoter (B cell expression), a runt transcription factor (CBFa2) promoter (stem cell specific expression), an CD14 promoter (monocytic cell expression), an CD43 promoter (leukocyte and platelet expression), an CD45 promoter (hematopoietic cell expression), an CD68 promoter (macrophage expression), a CYP450 3A4 promoter (hepatocyte expression), an desmin promoter (muscle expression), an elastase 1 promoter (pancreatic acinar cell expression, an endoglin promoter (endothelial cell expression), a fibroblast specific protein 1 promoter (FSP1) promoter (fibroblast cell expression), a fibronectin promoter (fibroblast cell expression), a fms-related tyrosine kinase 1 (FLT1) promoter (endothelial cell expression), a glial fibrillary acidic protein (GFAP) promoter (astrocyte expression), an insulin promoter (pancreatic beta cell expression), an integrin, alpha 2b (ITGA2B) promoter (megakaryocytes), an intracellular adhesion molecule 2 (ICAM-2) promoter (endothelial cells), an interferon beta (IFN-β) promoter (hematopoietic cells), a keratin 5 promoter (keratinocyte expression), a myoglobin (MB) promoter (muscle expression), a myogenic differentiation 1 (MYOD1) promoter (muscle expression), a nephrin promoter (podocyte expression), a bone gamma-carboxyglutamate protein 2 (OG-2) promoter (osteoblast expression), an 3-oxoacid CoA transferase 2B (Oxct2B) promoter, (haploid-spermatid expression), a surfactant protein B (SP-B) promoter (lung expression), a synapsin promoter (neuron expression), a Wiskott-Aldrich syndrome protein (WASP) promoter (hematopoietic cell expression).

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site specific DNA recombinase. According to certain embodiments, polynucleotides comprises at least one (typically two) site(s) for recombination mediated by a site specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, six, seven, eight, nine, ten or more.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The polynucleotides may comprise one or more recombination sites for any of a wide variety of site specific recombinases. It is to be understood that the target site for a site specific recombinase is in addition to any site(s) required for integration of a vector, e.g., a retroviral vector or lentiviral vector. As used herein, the terms “recombination sequence,” “recombination site,” or “site specific recombination site” refer to a particular nucleic acid sequence to which a recombinase recognizes and binds.

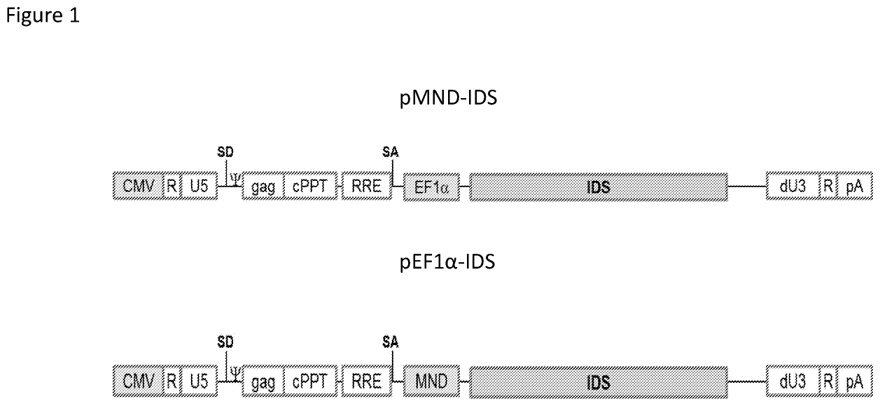

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lee and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F1, F2, F3 (Schlake and Bode, 1994), F4, F5 (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by φC31 homodimers (Groth et al., 2000). The product sites, attL and attR, are effectively inert to further φC31-mediated recombination (Belteki et al., 2003), making the reaction irreversible. For catalyzing insertions, it has been found that attB-bearing DNA inserts into a genomic attP site more readily than an attP site into a genomic attB site (Thyagaraj an et al., 2001; Belteki et al., 2003). Thus, typical strategies position by homologous recombination an attP-bearing “docking site” into a defined locus, which is then partnered with an attB-bearing incoming sequence for insertion.

In particular embodiments, to achieve efficient translation of each of the plurality of polypeptides, the polynucleotide sequences can be separated by one or more IRES sequences or polynucleotide sequences encoding self-cleaving polypeptides.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. Examples of IRES generally employed by those of skill in the art include those described in U.S. Pat. No. 6,692,736. Further examples of “IRES” known in the art include, but are not limited to IRES obtainable from picornavirus (Jackson et al., 1990) and IRES obtainable from viral or cellular mRNA sources, such as for example, immunoglobulin heavy-chain binding protein (BiP), the vascular endothelial growth factor (VEGF) (Huez et al. 1998. Mol. Cell. Biol. 18(11):6178-6190), the fibroblast growth factor 2 (FGF-2), and insulin-like growth factor (IGFII), the translational initiation factor eIF4G and yeast transcription factors TFIID and HAP4, the encephelomycarditis virus (EMCV) which is commercially available from Novagen (Duke et al., 1992. J. Virol 66(3):1602-9) and the VEGF IRES (Huez et al., 1998. Mol Cell Biol 18(11):6178-90). IRES have also been reported in viral genomes of Picornaviridae, Dicistroviridae and Flaviviridae species and in HCV, Friend murine leukemia virus (FrMLV) and Moloney murine leukemia virus (MoMLV).

In one embodiment, the IRES used in polynucleotides contemplated herein is an EMCV IRES.

In particular embodiments, the polynucleotides comprise polynucleotides that have a consensus Kozak sequence and that encode a desired polypeptide. As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO:17), where R is a purine (A or G) (Kozak, 1986. Cell. 44(2):283-92, and Kozak, 1987. Nucleic Acids Res. 15(20):8125-48).

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Efficient polyadenylation of the recombinant transcript is desirable as transcripts lacking a polyA tail are unstable and are rapidly degraded. Illustrative examples of polyA signals that can be used in a vector, includes an ideal polyA sequence (e.g., AATAAA, ATTAAA, AGTAAA), a bovine growth hormone polyA sequence (BGHpA), a rabbit β-globin polyA sequence (rβgpA), or another suitable heterologous or endogenous polyA sequence known in the art.

In some embodiments, a polynucleotide or cell harboring the polynucleotide utilizes a suicide gene, including an inducible suicide gene to reduce the risk of direct toxicity and/or uncontrolled proliferation. In specific embodiments, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID).

In certain embodiments, polynucleotides comprise gene segments that cause the genetically modified cells contemplated herein to be susceptible to negative selection in vivo. “Negative selection” refers to an infused cell that can be eliminated as a result of a change in the in vivo condition of the individual. The negative selectable phenotype may result from the insertion of a gene that confers sensitivity to an administered agent, for example, a compound. Negative selection genes are known in the art, and include, but are not limited to: the Herpes simplex virus type I thymidine kinase (HSV-I TK) gene which confers ganciclovir sensitivity; the cellular hypoxanthine phosphribosyltransferase (HPRT) gene, the cellular adenine phosphoribosyltransferase (APRT) gene, and bacterial cytosine deaminase.

In some embodiments, genetically modified cells comprise a polynucleotide further comprising a positive marker that enables the selection of cells of the negative selectable phenotype in vitro. The positive selectable marker may be a gene, which upon being introduced into the host cell, expresses a dominant phenotype permitting positive selection of cells carrying the gene. Genes of this type are known in the art, and include, but are not limited to hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

In one embodiment, the positive selectable marker and the negative selectable element are linked such that loss of the negative selectable element necessarily also is accompanied by loss of the positive selectable marker. In a particular embodiment, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See also the publications of PCT US91/08442 and PCT/US94/05601, by S. D. Lupton, describing the use of bifunctional selectable fusion genes derived from fusing a dominant positive selectable markers with negative selectable markers.

Preferred positive selectable markers are derived from genes selected from the group consisting of hph, nco, and gpt, and preferred negative selectable markers are derived from genes selected from the group consisting of cytosine deaminase, HSV-I TK, VZV TK, HPRT, APRT and gpt. Exemplary bifunctional selectable fusion genes contemplated in particular embodiments include, but are not limited to genes wherein the positive selectable marker is derived from hph or neo, and the negative selectable marker is derived from cytosine deaminase or a TK gene or selectable marker.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Illustrative examples of vectors include, but are not limited to plasmids (e.g., DNA plasmids or RNA plasmids), transposons, cosmids, bacterial artificial chromosomes, and viral vectors.

Illustrative methods of delivering polynucleotides contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

In preferred embodiments, polynucleotides encoding one or more therapeutic polypeptides, or fusion polypeptides may be introduced into a target cell by viral methods.

### E. Viral Vectors

Polynucleotides encoding one or more therapeutic polypeptides, or fusion polypeptides may be introduced into a target cell by non-viral or viral methods. In particular embodiments, polynucleotides encoding an I2S polypeptide are introduced into a target cell using a vector, preferably a viral vector, more preferably a retroviral vector, and even more preferably, a lentiviral vector.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a virus or viral particle that mediates nucleic acid transfer. Viral particles will typically include various viral components and sometimes also host cell components in addition to nucleic acid(s).

Illustrative examples of viral vector systems suitable for use in particular embodiments contemplated in particular embodiments include, but are not limited to adeno-associated virus (AAV), retrovirus, herpes simplex virus, adenovirus, vaccinia virus vectors for gene transfer.

Retroviruses are a common tool for gene delivery (Miller, 2000, Nature. 357: 455-460). As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus.” The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FM; bovine immune deficiency virus (BIV); and simian immunodeficiency virus (SIV). In one embodiment, HIV based vector backbones (i.e., HIV cis-acting sequence elements) are preferred. In particular embodiments, a lentivirus is used to deliver a polynucleotide encoding an I2S polypeptide to a cell.

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

In particular embodiments, the terms “lentiviral vector,” “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles and are present in DNA form in the DNA plasmids.

In various embodiments, a lentiviral vector contemplated herein comprises one or more LTRs, and one or more, or all, of the following accessory elements: a cPPT/FLAP, a Psi (T) packaging signal, an export element, a promoter operably linked to a polynucleotide encoding an I2S polypeptide, a poly (A) sequence, and may optionally comprise a WPRE or HPRE, an insulator element, a selectable marker, and a cell suicide gene, as discussed elsewhere herein.

In particular embodiments, lentiviral vectors contemplated herein may be integrative or non-integrating or integration defective lentivirus. As used herein, the term “integration defective lentivirus” or “refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

The term “long terminal repeat (LTR)” refers to domains of base pairs located at the ends of retroviral DNAs which, in their natural sequence context, are direct repeats and contain U3, R and U5 regions. The LTR contains numerous regulatory signals including transcriptional control elements, polyadenylation signals and sequences needed for replication and integration of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence,” “psi” and the symbol “Ψ,” refers to non-coding sequences located within the retroviral genome which are required for encapsidation of retroviral RNA strands during viral particle formation, see e.g., Clever et al., 1995. J. of Virology, Vol. 69, No. 4; pp. 2101-2109.

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. In a further embodiment, the 3′ LTR is modified such that the U5 region is replaced, for example, with an ideal poly(A) sequence. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. It should be noted that modifications to the LTRs such as modifications to the 3′ LTR, the 5′ LTR, or both 3′ and 5′ LTRs, are also included.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. During HIV-1 reverse transcription, central initiation of the plus-strand DNA at the central polypurine tract (cPPT) and central termination at the central termination sequence (CTS) lead to the formation of a three-stranded DNA structure: the HIV-1 central DNA flap. While not wishing to be bound by any theory, the DNA flap may act as a cis-active determinant of lentiviral genome nuclear import and/or may increase the titer of the virus. In particular embodiments, the retroviral or lentiviral vector backbones comprise one or more FLAP elements upstream or downstream of the heterologous genes of interest in the vectors. For example, in particular embodiments a transfer plasmid includes a FLAP element. In one embodiment, a vector comprises a FLAP element isolated from HIV-1. In another embodiment, a lentiviral vector contains a FLAP element with one or more mutations in the cPPT and/or CTS elements. In yet another embodiment, a lentiviral vector comprises either a cPPT or CTS element. In yet another embodiment, a lentiviral vector does not comprise a cPPT or CTS element.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766). In particular embodiments, vectors comprise a posttranscriptional regulatory element such as a WPRE or HPRE. In particular embodiments, vectors lack or do not comprise a posttranscriptional regulatory element such as a WPRE or HPRE.

Elements directing the efficient termination and polyadenylation of the heterologous nucleic acid transcripts increases heterologous gene expression. Illustrative examples of polyA signals that can be used in a vector, includes an ideal polyA sequence (e.g., AATAAA, ATTAAA, AGTAAA), a bovine growth hormone polyA sequence (BGHpA), a rabbit β-globin polyA sequence (rβgpA), or another suitable heterologous or endogenous polyA sequence known in the art.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid.

In particular embodiments, a retroviral vector comprises a left (5′) lentiviral LTR; a Psi (w) packaging signal; a retroviral export element; a cPPT/FLAP; a promoter operably linked to a polynucleotide encoding iduronate 2-sulfatase (I2S) polypeptide; and a right (3′) lentiviral LTR. In certain embodiments, the retroviral vector is preferably a lentiviral vector, more preferably an HIV lentiviral vector, and even preferably, an HIV-1 lentiviral vector.

In particular embodiments, a lentiviral vector comprises a left (5′) lentiviral LTR wherein the promoter region of the LTR is replaced with a heterologous promoter; a Psi (w) packaging signal; a retroviral export element; a cPPT/FLAP; a promoter operably linked to a polynucleotide encoding iduronate 2-sulfatase (12S) polypeptide; and a right (3′) lentiviral LTR. In certain embodiments, the heterologous promoter is a cytomegalovirus (CMV) promoter, a Rous Sarcoma Virus (RSV) promoter, or a Simian Virus 40 (SV40) promoter.

In particular embodiments, a lentiviral vector comprises a left (5′) lentiviral LTR; a Psi (ψ) packaging signal; a retroviral export element; a cPPT/FLAP; a promoter operably linked to a polynucleotide encoding iduronate 2-sulfatase (I2S) polypeptide; and a right (3′) lentiviral LTR that comprises one or more modification compared to an unmodified LTR. In certain embodiments, the 3′ LTR preferably comprises one or more deletions that prevent viral transcription beyond the first round of viral replication, more preferably comprises a deletion of the TATA box and Sp1 and NF-κB transcription factor binding sites in the U3 region of the 3′ LTR, and even more preferably is a self-inactivating (SIN) LTR.

In particular embodiments, a lentiviral vector comprises a left (5′) lentiviral LTR wherein the promoter region of the LTR is replaced with a heterologous promoter; a Psi (w) packaging signal; a retroviral export element; a cPPT/FLAP; a promoter operably linked to a polynucleotide encoding iduronate 2-sulfatase (I2S) polypeptide; and a right (3′) lentiviral SIN LTR.

In particular embodiments, a lentiviral vector comprises a left (5′) lentiviral LTR wherein the promoter region of the LTR is replaced with a heterologous promoter; a Psi (w) packaging signal; a retroviral export element; a cPPT/FLAP; a myeloproliferative sarcoma virus enhancer, negative control region deleted, d1587rev primer-binding site substituted (MND) promoter or transcriptionally active fragment thereof operably linked to a polynucleotide encoding a human iduronate 2-sulfatase (I2S) polypeptide; and a right (3′) lentiviral SIN LTR.

In particular embodiments, a lentiviral vector comprises a left (5′) lentiviral LTR wherein the promoter region of the LTR is replaced with a heterologous promoter; a Psi (w) packaging signal; a retroviral export element; a cPPT/FLAP; an elongation factor 1 alpha (EF1a) promoter or transcriptionally active fragment thereof operably linked to a polynucleotide encoding a human iduronate 2-sulfatase (I2S) polypeptide; and a right (3′) lentiviral SIN LTR. In preferred embodiments, the EF1α promoter lacks the first intron of the human EF1α gene and is referred to as an “EF1α short promoter.” In other embodiments, the EF1α promoter comprises the first intron of the human EF1α gene and is referred to as an “EF1α long promoter.”

In particular embodiments, a lentiviral vector comprises a left (5′) CMV promoter/HIV-1 chimeric LTR; a Psi (w) packaging signal; an RRE retroviral export element; a cPPT/FLAP; an MND promoter or EF1α-short promoter operably linked to a polynucleotide encoding a human iduronate 2-sulfatase (I2S) polypeptide; and a right (3′) lentiviral SIN LTR.

In particular embodiments, a lentiviral vector comprises a left (5′) CMV promoter/HIV-1 chimeric LTR; a Psi (w) packaging signal; an RRE retroviral export element; a cPPT/FLAP; an MND promoter or EF1α-short promoter operably linked to a polynucleotide encoding a human iduronate 2-sulfatase (I2S) polypeptide; a right (3′) lentiviral SIN LTR; and a heterologous polyadenylation signal. In certain embodiments, the polyadenylation signal is an artificial polyadenylation signal, a bovine growth hormone polyadenylation signal or a rabbit β-globin polyadenylation signal.

Large scale viral particle production is often necessary to achieve a reasonable viral titer. Viral particles are produced by transfecting a transfer vector into a packaging cell that comprises viral structural and/or accessory genes, e.g., gag, pol, env, tat, rev, vif, vpr, vpu, vpx, or nef genes or other retroviral genes.

As used herein, the term “packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are well known by those of skill in the art. A retroviral/lentiviral transfer vector can be introduced into a packaging cell line, via transfection, transduction or infection, to generate a producer cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120. Preferably, the viral env proteins expressed by packaging cells are encoded on a separate vector from the viral gag and pol genes, as has been previously described.

Illustrative examples of retroviral-derived env genes which can be employed in particular embodiments include, but are not limited to: MLV envelopes, 10A1 envelope, BAEV, FeLV-B, RD114, SSAV, Ebola, Sendai, FPV (Fowl plague virus), and influenza virus envelopes. Similarly, genes encoding envelopes from RNA viruses (e.g., RNA virus families of Picornaviridae, Calciviridae, Astroviridae, Togaviridae, Flaviviridae, Coronaviridae, Paramyxoviridae, Rhabdoviridae, Filoviridae, Orthomyxoviridae, Bunyaviridae, Arenaviridae, Reoviridae, Birnaviridae, Retroviridae) as well as from the DNA viruses (families of Hepadnaviridae, Circoviridae, Parvoviridae, Papovaviridae, Adenoviridae, Herpesviridae, Poxyiridae, and Iridoviridae) may be utilized. Representative examples of these viruses include, but are not limited to, FeLV, VEE, HFVW, WDSV, SFV, Rabies, ALV, BIV, BLV, EBV, CAEV, SNV, ChTLV, STLV, MPMV, SMRV, RAV, FuSV, WE, AEV, AMV, CT10, and EIAV.

In other embodiments, envelope proteins for pseudotyping a virus include, but are not limited to any of the following virus: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Human immunodeficiency virus (HIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), West Nile virus, any encephaliltis causing virus.

In one embodiment, packaging cells are provided, which produce recombinant retrovirus, e.g., lentivirus, pseudotyped with the VSV-G glycoprotein.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells. In a preferred embodiment, lentiviral envelope proteins are pseudotyped with VSV-G. In one embodiment, packaging cells are provided which produce recombinant retrovirus, e.g., lentivirus, pseudotyped with the VSV-G envelope glycoprotein.

As used herein, the term “packaging cell lines” is used in reference to cell lines that do not contain a packaging signal, but do stably or transiently express viral structural proteins and replication enzymes (e.g., gag, pol and env) which are necessary for the correct packaging of viral particles. Any suitable cell line can be employed to prepare packaging cells. Generally, the cells are mammalian cells. In a particular embodiment, the cells used to produce the packaging cell line are human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In preferred embodiments, the packaging cells are 293 cells, 293T cells, or A549 cells. In another preferred embodiment, the cells are A549 cells.

As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Methods of preparing viral stock solutions are known in the art and are illustrated by, e.g., Y. Soneoka et al. (1995) Nucl. Acids Res. 23:628-633, and N. R. Landau et al. (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

In particular embodiments, host cells transduced with viral vector that expresses one or more polypeptides to generate genetically modified cells that are administered to a subject to treat and/or prevent and/or ameliorate at least one symptom of Hunter Syndrome. Other methods relating to the use of viral vectors in gene therapy, which may be utilized according to certain embodiments, can be found in, e.g., Kay, M. A. (1997) Chest 111(6 Supp.):138S-142S; Ferry, N. and Heard, J. M. (1998) Hum. Gene Ther. 9:1975-81; Shiratory, Y. et al. (1999) Liver 19:265-74; Oka, K. et al. (2000) Curr. Opin. Lipidol. 11:179-86; Thule, P. M. and Liu, J. M. (2000) Gene Ther. 7:1744-52; Yang, N. S. (1992) Crit. Rev. Biotechnol. 12:335-56; Alt, M. (1995) J. Hepatol. 23:746-58; Brody, S. L. and Crystal, R. G. (1994) Ann. N.Y. Acad. Sci. 716:90-101; Strayer, D. S. (1999) Expert Opin. Investig. Drugs 8:2159-2172; Smith-Arica, J. R. and Bartlett, J. S. (2001) Curr. Cardiol. Rep. 3:43-49; and Lee, H. C. et al. (2000) Nature 408:483-8.

A “host cell” includes cells transfected, infected, or transduced in vivo, ex vivo, or in vitro with a recombinant vector or a polynucleotide contemplated herein. Host cells may include packaging cells, producer cells, and cells infected with viral vectors. In particular embodiments, host cells infected with viral vector of the invention are administered to a subject in need of therapy. In certain embodiments, the term “target cell” is used interchangeably with host cell and refers to transfected, infected, or transduced cells of a desired cell type. In preferred embodiments, the target cell is a stem cell or progenitor cell. In certain preferred embodiments, the target cell is a somatic cell, e.g., adult stem cell, progenitor cell, or differentiated cell. In particular preferred embodiments, the target cell is a hematopoietic cell, e.g., a hematopoietic stem or progenitor cell, or CD34⁺ cell. Further therapeutic target cells are discussed, herein.

### F. Genetically Modified Cells

In various embodiments, cells are genetically modified to express an I2S polypeptide, and the genetically modified cells are used to treat neuronal ceroid lipofuscinoses. The cells may be genetically modified ex vivo, in vitro, or ex vivo. As used herein, the term “genetically engineered” or “genetically modified” refers to the addition of extra genetic material in the form of DNA or RNA into the total genetic material in a cell. The terms, “genetically modified cells,” “modified cells,” and, “genetically engineered cells,” are used interchangeably. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g, I2S.

The cells can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are allogeneic.

In particular embodiments, vectors encoding 12S are introduced into one or more animal cells, preferably a mammal, e.g., a non-human primate or human, and more preferably a human.

In certain embodiments, a population of cells is transduced with a vector contemplated herein. As used herein, the term “population of cells” refers to a plurality of cells that may be made up of any number and/or combination of homogenous or heterogeneous cell types, as described elsewhere herein. For example, for transduction of hematopoietic stem or progenitor cells, a population of cells may be isolated or obtained from umbilical cord blood, placental blood, bone marrow, or peripheral blood. A population of cells may comprise about 10%, about 20%, about 30%, about 40%, about 50%, about 60%, about 70%, about 80%, about 90%, or about 100% of the target cell type to be transduced. In certain embodiments, hematopoietic stem or progenitor cells may be isolated or purified from a population of heterogeneous cells using methods known in the art.

In particular embodiments, the cell is a primary cell. The term “primary cell” as used herein is known in the art to refer to a cell that has been isolated from a tissue and has been established for growth in vitro or ex vivo. Corresponding cells have undergone very few, if any, population doublings and are therefore more representative of the main functional component of the tissue from which they are derived in comparison to continuous cell lines, thus representing a more representative model to the in vivo state. Methods to obtain samples from various tissues and methods to establish primary cell lines are well-known in the art (see, e.g., Jones and Wise, Methods Mol Biol. 1997). Primary cells for use in the method of the invention are derived from, e.g., blood, lymphoma and epithelial tumors. In one embodiment, the primary cell is a hematopoietic stem or progenitor cell.

The term “stem cell” refers to a cell which is an undifferentiated cell capable of (1) long term self-renewal, or the ability to generate at least one identical copy of the original cell, (2) differentiation at the single cell level into multiple, and in some instance only one, specialized cell type and (3) of in vivo functional regeneration of tissues. Stem cells are subclassified according to their developmental potential as totipotent, pluripotent, multipotent and oligo/unipotent. “Self-renewal” refers a cell with a unique capacity to produce unaltered daughter cells and to generate specialized cell types (potency). Self-renewal can be achieved in two ways. Asymmetric cell division produces one daughter cell that is identical to the parental cell and one daughter cell that is different from the parental cell and is a progenitor or differentiated cell. Symmetric cell division produces two identical daughter cells. “Proliferation” or “expansion” of cells refers to symmetrically dividing cells.

As used herein, the term “progenitor” or “progenitor cells” refers to cells have the capacity to self-renew and to differentiate into more mature cells. Many progenitor cells differentiate along a single lineage, but may have quite extensive proliferative capacity.

Hematopoietic stem cells (HSCs) give rise to committed hematopoietic progenitor cells (HPCs) that are capable of generating the entire repertoire of mature blood cells over the lifetime of an organism. The term “hematopoietic stem cell” or “HSC” refers to multipotent stem cells that give rise to the all the blood cell types of an organism, including myeloid (e.g., monocytes and macrophages, neutrophils, basophils, eosinophils, erythrocytes, megakaryocytes/platelets, dendritic cells), and lymphoid lineages (e.g., T-cells, B-cells, NK-cells), and others known in the art (See Fei, R., et al., U.S. Pat. No. 5,635,387; McGlave, et al., U.S. Pat. No. 5,460,964; Simmons, P., et al., U.S. Pat. No. 5,677,136; Tsukamoto, et al., U.S. Pat. No. 5,750,397; Schwartz, et al., U.S. Pat. No. 5,759,793; DiGuisto, et al., U.S. Pat. No. 5,681,599; Tsukamoto, et al., U.S. Pat. No. 5,716,827). In one embodiment, the HSC is a CD34⁺ cell. When transplanted into lethally irradiated animals or humans, hematopoietic stem and progenitor cells can repopulate the erythroid, neutrophil-macrophage, megakaryocyte and lymphoid hematopoietic cell pool.

Preferred target cell types transduced with the compositions and methods contemplated herein include, hematopoietic cells, preferably human hematopoietic cells, more preferably human hematopoietic stem and progenitor cells, and even more preferably CD34⁺ human hematopoietic stem cells.

Illustrative sources to obtain hematopoietic cells transduced with the methods and compositions contemplated herein include, but are not limited to: cord blood, bone marrow or mobilized peripheral blood.

In particular embodiments, hematopoietic cells transduced with viral vectors encoding I2S contemplated herein include CD34⁺ cells. The term “CD34⁺ cell,” as used herein refers to a cell expressing the CD34 protein on its cell surface. “CD34,” as used herein refers to a cell surface glycoprotein (e.g., sialomucin protein) that often acts as a cell-cell adhesion factor. CD34⁺ is a cell surface marker of both hematopoietic stem and progenitor cells.

Additional illustrative examples of hematopoietic stem or progenitor cells suitable for transduction with the methods and compositions contemplated herein include hematopoietic cells that are CD34⁺CD38^{Lo}CD90⁺CD45^{RA-}, hematopoietic cells that are CD34⁺, CD59⁺, Thy1/CD90⁺, CD38^{Lo/-}, C-kit/CD117⁺, and Line, and hematopoietic cells that are CD133⁺.

In one embodiment, hematopoietic cells transduced with viral vectors encoding I2S contemplated herein include CD34⁺CD133⁺ cells.

Various methods exist to characterize hematopoietic hierarchy. One method of characterization is the SLAM code. The SLAM (Signaling lymphocyte activation molecule) family is a group of >10 molecules whose genes are located mostly tandemly in a single locus on chromosome 1 (mouse), all belonging to a subset of immunoglobulin gene superfamily, and originally thought to be involved in T-cell stimulation. This family includes CD48, CD150, CD244, etc., CD150 being the founding member, and, thus, also called slamF1, i.e., SLAM family member 1. The signature SLAM code for the hematopoietic hierarchy is hematopoietic stem cells (HSC)-CD150⁺CD48⁻CD244⁻; multipotent progenitor cells (MPPs)-CD150⁻CD48⁻CD244⁺; lineage-restricted progenitor cells (LRPs)-CD150⁻CD48⁺CD244⁺; common myeloid progenitor (CMP)-lin-SCA-1-c-kit⁺CD34⁺CD16/32^{mid}; granulocyte-macrophage progenitor (GMP)-lin⁻SCA-1-c-kit⁺CD34⁺CD16/32^{hi}; and megakaryocyte-erythroid progenitor (MEP)-lin⁻SCA-1-c-kit⁺CD34⁻CD16/32^{low}.

In one embodiment, hematopoietic cells transduced with viral vectors encoding 12S contemplated herein include CD150⁺CD48⁻CD244⁻ cells.

In various embodiments, a population of hematopoietic cells comprising hematopoietic stem and progenitor cells (HSPCs) transduced with a viral vector encoding I2S as contemplated herein is provided. In preferred embodiments, the HSPCs are CD34⁺ hematopoietic cells.

### G. Compositions and Formulations

The compositions and formulations contemplated herein may comprise a combination of any number of transduced or non-transduced cells or a combination thereof, viral vectors, polypeptides, and polynucleotides contemplated herein. Compositions include, but are not limited to pharmaceutical compositions. A “pharmaceutical composition” refers to a composition formulated with a pharmaceutically-acceptable carrier for administration to a cell or an animal, either alone, or in combination with one or more other modalities of therapy. It will also be understood that, if desired, the compositions may be administered in combination with other agents as well, such as, e.g., cytokines, growth factors, hormones, small molecules, pro-drugs, drugs, antibodies, or other various pharmaceutically-active agents. In particular embodiments, there is virtually no limit to other components that may also be included in the compositions, provided that the additional agents do not adversely affect the ability of the composition to deliver the intended therapy.

Particular ex vivo and in vitro formulations and compositions contemplated herein may comprise a combination of transduced or non-transduced cells or a combination thereof, and viral vectors formulated with a pharmaceutically-acceptable carrier for administration to a cell, tissue, organ, or an animal, either alone, or in combination with one or more other modalities of therapy.

Particular in vivo formulations and compositions contemplated herein may comprise a combination of viral vectors formulated with a pharmaceutically-acceptable carrier for administration to a cell, tissue, organ, or an animal, either alone, or in combination with one or more other modalities of therapy.

In certain embodiments, compositions contemplated herein comprise a population of cells, comprising a therapeutically-effective amount of transduced cells, e.g., hematopoietic cells, hematopoietic stem cells, hematopoietic progenitor cells, CD34⁺ cells, CD133⁺ cells, etc., formulated with one or more pharmaceutically acceptable carriers.

In certain other embodiments, the present invention provides compositions comprising a retroviral vector, e.g., a lentiviral vector formulated with one or more pharmaceutically acceptable carriers.

Pharmaceutical compositions contemplated herein comprise transduced cells comprising a vector or provirus encoding I2S as contemplated herein and a pharmaceutically acceptable carrier.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” refers to a diluent, adjuvant, excipient, or vehicle with which the therapeutic cells are administered. Illustrative examples of pharmaceutical carriers can be sterile liquids, such as cell culture media, water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients in particular embodiments, include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Except insofar as any conventional media or agent is incompatible with the active ingredient, its use in the therapeutic compositions is contemplated. Supplementary active ingredients can also be incorporated into the compositions.

In one embodiment, a composition comprising a pharmaceutically acceptable carrier is suitable for administration to a subject. In particular embodiments, a composition comprising a carrier is suitable for parenteral administration, e.g., intravascular (intravenous or intraarterial), intraperitoneal or intramuscular administration. In particular embodiments, a composition comprising a pharmaceutically acceptable carrier is suitable for intraventricular, intraspinal, or intrathecal administration. Pharmaceutically acceptable carriers include sterile aqueous solutions, cell culture media, or dispersions. The use of such media and agents for pharmaceutically active substances is well known in the art. Except insofar as any conventional media or agent is incompatible with the transduced cells, use thereof in the pharmaceutical compositions is contemplated.

In particular embodiments, compositions contemplated herein comprise genetically modified hematopoietic stem and/or progenitor cells and a pharmaceutically acceptable carrier. A composition comprising a cell-based composition contemplated herein can be administered separately by enteral or parenteral administration methods or in combination with other suitable compounds to effect the desired treatment goals

The pharmaceutically acceptable carrier must be of sufficiently high purity and of sufficiently low toxicity to render it suitable for administration to the human subject being treated. It further should maintain or increase the stability of the composition. The pharmaceutically acceptable carrier can be liquid or solid and is selected, with the planned manner of administration in mind, to provide for the desired bulk, consistency, etc., when combined with other components of the composition. For example, the pharmaceutically acceptable carrier can be, without limitation, a binding agent (e.g., pregelatinized maize starch, polyvinylpyrrolidone or hydroxypropyl methylcellulose, etc.), a filler (e.g., lactose and other sugars, microcrystalline cellulose, pectin, gelatin, calcium sulfate, ethyl cellulose, polyacrylates, calcium hydrogen phosphate, etc.), a lubricant (e.g., magnesium stearate, talc, silica, colloidal silicon dioxide, stearic acid, metallic stearates, hydrogenated vegetable oils, corn starch, polyethylene glycols, sodium benzoate, sodium acetate, etc.), a disintegrant (e.g., starch, sodium starch glycolate, etc.), or a wetting agent (e.g., sodium lauryl sulfate, etc.). Other suitable pharmaceutically acceptable carriers for the compositions contemplated herein include, but are not limited to, water, salt solutions, alcohols, polyethylene glycols, gelatins, amyloses, magnesium stearates, talcs, silicic acids, viscous paraffins, hydroxymethylcelluloses, polyvinylpyrrolidones and the like.

Such carrier solutions also can contain buffers, diluents and other suitable additives. The term “buffer” as used herein refers to a solution or liquid whose chemical makeup neutralizes acids or bases without a significant change in pH. Examples of buffers contemplated herein include, but are not limited to, Dulbecco's phosphate buffered saline (PBS), Ringer's solution, 5% dextrose in water (D5W), normal/physiologic saline (0.9% NaCl).

The pharmaceutically acceptable carriers may be present in amounts sufficient to maintain a pH of the composition of about 7. Alternatively, the composition has a pH in a range from about 6.8 to about 7.4, e.g., 6.8, 6.9, 7.0, 7.1, 7.2, 7.3, and 7.4. In still another embodiment, the composition has a pH of about 7.4.

Compositions contemplated herein may comprise a nontoxic pharmaceutically acceptable medium. The compositions may be a suspension. The term “suspension” as used herein refers to non-adherent conditions in which cells are not attached to a solid support. For example, cells maintained as a suspension may be stirred or agitated and are not adhered to a support, such as a culture dish.

In particular embodiments, compositions contemplated herein are formulated in a suspension, where the hematopoietic stem and/or progenitor cells are dispersed within an acceptable liquid medium or solution, e.g., saline or serum-free medium, in an intravenous (IV) bag or the like. Acceptable diluents include, but are not limited to water, PlasmaLyte, Ringer's solution, isotonic sodium chloride (saline) solution, serum-free cell culture medium, and medium suitable for cryogenic storage, e.g., Cryostor® medium.

In certain embodiments, a pharmaceutically acceptable carrier is substantially free of natural proteins of human or animal origin, and suitable for storing a composition comprising a population of cells, e.g., hematopoietic stem and progenitor cells. The therapeutic composition is intended to be administered into a human patient, and thus is substantially free of cell culture components such as bovine serum albumin, horse serum, and fetal bovine serum.

In some embodiments, compositions are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In a preferred embodiment, the compositions comprising hematopoietic stem and/or progenitor cells are formulated in PlasmaLyte.

In various embodiments, compositions comprising hematopoietic stem and/or progenitor cells are formulated in a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In one embodiment, the compositions are formulated in a solution comprising 50:50 PlasmaLyte A to CryoStor CS10.

In particular embodiments, the composition is substantially free of mycoplasma, endotoxin, and microbial contamination. By “substantially free” with respect to endotoxin is meant that there is less endotoxin per dose of cells than is allowed by the FDA for a biologic, which is a total endotoxin of 5 EU/kg body weight per day, which for an average 70 kg person is 350 EU per total dose of cells. In particular embodiments, compositions comprising hematopoietic stem or progenitor cells transduced with a retroviral vector contemplated herein contains about 0.5 EU/mL to about 5.0 EU/mL, or about 0.5 EU/mL, 1.0 EU/mL, 1.5 EU/mL, 2.0 EU/mL, 2.5 EU/mL, 3.0 EU/mL, 3.5 EU/mL, 4.0 EU/mL, 4.5 EU/mL, or 5.0 EU/mL.

In certain embodiments, compositions and formulations suitable for the delivery of viral vector systems (i.e., viral-mediated transduction) are contemplated including, but not limited to, retroviral (e.g., lentiviral) vectors.

Exemplary formulations for ex vivo delivery may also include the use of various transfection agents known in the art, such as calcium phosphate, electroporation, heat shock and various liposome formulations (i.e., lipid-mediated transfection). Liposomes, as described in greater detail below, are lipid bilayers entrapping a fraction of aqueous fluid. DNA spontaneously associates to the external surface of cationic liposomes (by virtue of its charge) and these liposomes will interact with the cell membrane.

In particular embodiments, formulation of pharmaceutically-acceptable carrier solutions is well-known to those of skill in the art, as is the development of suitable dosing and treatment regimens for using the particular compositions described herein in a variety of treatment regimens, including e.g., enteral and parenteral, e.g., intravascular, intravenous, intrarterial, intraosseously, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, 20th Edition. Baltimore, Md.: Lippincott Williams & Wilkins, 2005, which is incorporated by reference herein, in its entirety.

### H. Gene Therapy Methods

The genetically modified cells contemplated herein provide improved drug products for use in the prevention, treatment, and amelioration of Hunter Syndrome or for preventing, treating, or ameliorating at least one symptom associated with Hunter Syndrome or a subject having a mutation in an 12S gene that decreases or abolishes 12S expression and/or activity.

As used herein, the term “drug product” refers to genetically modified cells produced using the compositions and methods contemplated herein. In particular embodiments, the drug product comprises genetically modified hematopoietic stem or progenitor cells, e.g., CD34⁺ cells. Without wishing to be bound to any particular theory, increasing the amount of a therapeutic gene in a drug product may allow treatment of subjects having no or minimal expression of the corresponding gene in vivo, thereby significantly expanding the opportunity to bring gene therapy to subjects for which gene therapy was not previously a viable treatment option.

The transduced cells and corresponding retroviral vectors contemplated herein provide improved methods of gene therapy. As used herein, the term “gene therapy” refers to the introduction of a gene into a cell's genome. In various embodiments, a viral vector of the invention comprises an expression control sequence that expresses a therapeutic transgene encoding a polypeptide that provides curative, preventative, or ameliorative benefits to a subject diagnosed with or that is suspected of having Hunter Syndrome, or a subject having I2S gene comprising one or more mutations that decrease I2S expression and/or activity.

In various embodiments, the retroviral vectors are administered by direct injection to a cell, tissue, or organ of a subject in need of gene therapy, in vivo. In various other embodiments, cells are transduced in vitro or ex vivo with vectors contemplated herein, and optionally expanded ex vivo. The transduced cells are then administered to a subject in need of gene therapy.

Cells suitable for transduction and administration in the gene therapy methods contemplated herein include, but are not limited to stem cells, progenitor cells, and differentiated cells as described elsewhere herein. In certain embodiments, the transduced cells are hematopoietic stem or progenitor cells as described elsewhere herein.

Preferred cells for use in the gene therapy compositions and methods contemplated herein include autologous/autogeneic (“self”) cells.

As used herein, the terms “individual” and “subject” are often used interchangeably and refer to any animal that exhibits a symptom of a disease, disorder, or condition that can be treated with the gene therapy vectors, cell-based therapeutics, and methods contemplated elsewhere herein. In preferred embodiments, a subject includes any animal that exhibits symptoms of a neuronal ceroid lipofuscinoses that can be treated with the gene therapy vectors, cell-based therapeutics, and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have Hunter Syndrome, have been diagnosed with Hunter Syndrome, or are at risk or having Hunter Syndrome.

As used herein, the term “patient” refers to a subject that has been diagnosed with a particular disease, disorder, or condition that can be treated with the gene therapy vectors, cell-based therapeutics, and methods disclosed elsewhere herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated. In particular embodiments, the disease or condition being treated is Hunter Syndrome, wherein the at least one symptom is selected from the group consisting of: build up of GAGs, thickening of organ and tissues, difficulty breathing, difficulty swallowing, joint stiffness, cognitive function decline, and motor function decline.

In particular embodiments, a subject is administered an amount of genetically modified cell or gene therapy vector sufficient to treat, prevent, or ameliorate at least one symptom of Hunter Syndrome.

As used herein, the term “amount” refers to “an amount effective” or “an effective amount” of a virus or transduced therapeutic cell to achieve a beneficial or desired prophylactic or therapeutic result, including clinical results.

A “prophylactically effective amount” refers to an amount of a virus or transduced therapeutic cell effective to achieve the desired prophylactic result. Typically but not necessarily, since a prophylactic dose is used in subjects prior to or at an earlier stage of disease, the prophylactically effective amount is less than the therapeutically effective amount.

A “therapeutically effective amount” of a virus or transduced therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient).

Without wishing to be bound to any particular theory, an important advantage provided by the vectors, compositions, and methods of the present invention is the high efficacy of gene therapy that can be achieved by administering populations of cells comprising high percentages of transduced cells compared to existing methods.

The transduced cells may be administered as part of a bone marrow or cord blood transplant in an individual that has or has not undergone bone marrow ablative therapy. In one embodiment, transduced cells of the invention are administered in a bone marrow transplant to an individual that has undergone chemoablative or radioablative bone marrow therapy.

In one embodiment, a dose of transduced cells is delivered to a subject intravenously. In preferred embodiments, transduced hematopoietic stem cells are intravenously administered to a subject.

In one illustrative embodiment, the effective amount of transduced cells provided to a subject is at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, or at least 10×10⁶ cells/kg, or more cells/kg, including all intervening doses of cells.

In another illustrative embodiment, the effective amount of transduced cells provided to a subject is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, or about 10×10⁶ cells/kg, or more cells/kg, including all intervening doses of cells.

In another illustrative embodiment, the effective amount of transduced cells provided to a subject is from about 2×10⁶ cells/kg to about 10×10⁶ cells/kg, about 3×10⁶ cells/kg to about 10×10⁶ cells/kg, about 4×10⁶ cells/kg to about 10×10⁶ cells/kg, about 5×10⁶ cells/kg to about 10×10⁶ cells/kg, 2×10⁶ cells/kg to about 6×10⁶ cells/kg, 2×10⁶ cells/kg to about 7×10⁶ cells/kg, 2×10⁶ cells/kg to about 8×10⁶ cells/kg, 3×10⁶ cells/kg to about 6×10⁶ cells/kg, 3×10⁶ cells/kg to about 7×10⁶ cells/kg, 3×10⁶ cells/kg to about 8×10⁶ cells/kg, 4×10⁶ cells/kg to about 6×10⁶ cells/kg, 4×10⁶ cells/kg to about 7×10⁶ cells/kg, 4×10⁶ cells/kg to about 8×10⁶ cells/kg, 5×10⁶ cells/kg to about 6×10⁶ cells/kg, 5×10⁶ cells/kg to about 7×10⁶ cells/kg, 5×10⁶ cells/kg to about 8×10⁶ cells/kg, or 6×10⁶ cells/kg to about 8×10⁶ cells/kg, including all intervening doses of cells.

In certain embodiments, it can generally be stated that a pharmaceutical composition comprising the genetically modified cells described herein may be administered at a dosage of 10² to 10¹⁰ cells/kg body weight, preferably 10⁵ to 10⁶ cells/kg body weight, including but not limited to 1×10⁶ cells/mL, 2×10⁶ cells/mL, 3×10⁶ cells/mL, 4×10⁶ cells/mL, 5×10⁶ cells/mL, 6×10⁶ cells/mL, 7×10⁶ cells/mL, 8×10⁶ cells/mL, 9×10⁶ cells/mL, 10×10⁶ cells/mL, and all integer values within those ranges. The number of cells will depend upon the ultimate use for which the composition is intended as will the type of cells included therein. For uses provided in some embodiments, the cells are generally in a volume of a liter or less, can be 500 mLs or less, even 250 mLs or 100 mLs or less. Hence the density of the desired cells in particular embodiments is typically greater than 10⁶ cells/mL, 10⁷ cells/mL, or 10⁸ cells/mL. The clinically relevant number of cells can be apportioned into multiple infusions that cumulatively equal or exceed 10⁵, 10⁶, 10⁷, 10⁸, 10⁹, 10¹⁰ or 10¹² cells. Cell-based compositions may be administered multiple times at dosages within these ranges. The cells may be allogeneic, syngeneic, xenogeneic, or autologous to the patient undergoing therapy.

Some variation in dosage will necessarily occur depending on the condition of the subject being treated. The person responsible for administration will, in any event, determine the appropriate dose for the individual subject.

One of ordinary skill in the art would be able to use routine methods in order to determine the appropriate route of administration and the correct dosage of an effective amount of a composition comprising transduced cells or gene therapy vectors contemplated herein.

In particular embodiments, multiple administrations of pharmaceutical compositions contemplated herein may be required to effect therapy. In particular embodiments, the drug product is administered once. In certain embodiments, the drug product is administered 1, 2, 3, 4, 5, 6, 7, 8, 9, or 10 or more times over a span of 1 year, 2 years, 5, years, 10 years, or more.

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Construction of I2S Vectors

Third generation lentiviral vectors containing a chimeric 5′ LTR; a myeloproliferative sarcoma virus enhancer, negative control region deleted, d1587rev primer-binding site substituted (MND) promoter or a short elongation factor 1 alpha (EF1a) promoter; a polynucleotide encoding iduronate 2-sulfatase (I2S) polypeptide; and a self-inactivating (SIN) 3′ LTR were constructed. See e.g., FIG. 1 and SEQ ID NOs: 1 and 2. Tables 1 and 2 show the Identity, Genbank Reference, Source Name and Citation for the various nucleotide segments of exemplary lentiviral vectors encoding I2S.

TABLE 1
pMND-I2S LVV

| | | GenBank | | |
|---|---|---|---|---|
| Nucleotides | Identity | Reference | Source Name | Citation |
| 1-185 | pUC19 plasmid | Accession | pUC19 | New England |
| | backbone | #L09137.2 | | Biolabs |
| | | nt 1-185 | | (Attachment 1) |
| 185-222 | Linker | Not applicable | Synthetic | Not applicable¹ |
| 223-800 | CMV | Not Applicable | pHCMV | (1994) PNAS 91: |
| | | | | 9564-68 |
| 801-1136 | R, U5, PBS, and | Accession | pNL4-3 | Maldarelli, |
| | packaging | #M19921.2 | | et. al. (1991) |
| | sequences | nt 454-789 | | J Virol: |
| | | | | 65(11): 5732-43 |
| 1137-1139 | Gag start codon | Not Applicable¹ | Synthetic | Not applicable |
| | (ATG) changed | | | |
| | to stop codon | | | |
| | (TAG) | | | |
| 1140-1240 | HIV-1 gag | Accession | pNL4-3 | Maldarelli, |
| | sequence | #M19921.2 | | et. al. (1991) |
| | | nt 793-893 | | J Virol: |
| | | | | 65 (11): 5732-43 |
| 1241-1243 | HIV-1 gag | Not Applicable | Synthetic | Not applicable |
| | sequence changed | | | |
| | to a second stop | | | |
| | codon | | | |
| 1244-1595 | HIV-1 gag | Accession | pNL4-3 | Maldarelli, |
| | sequence | #M19921.2 | | et. al. (1991) |
| | | nt 897-1248 | | J Virol: |
| | | | | 65(11): 5732-43 |
| 1596-1992 | HIV-1 pol | Accession | pNL4-3 | Maldarelli, |
| | cPPT/CTS | #M19921.2 | | et. al. (1991) |
| | | nt 4745-5125 | | J Virol: |
| | | | | 65(11): 5732-43 |
| 1993-2517 | HIV-1, isolate | Accession | PgTAT-CMV | Malim, M. H. |
| | HXB3 env region | #M14100.1 | | Nature (1988) |
| | (RRE) | nt 1875-2399 | | 335: 181-183 |
| 2518-2693 | HIV-1 env | Accession | pNL4-3 | Maldarelli, |
| | sequences S/A | #M19921.2 | | et. al. (1991) |
| | | nt 8290-8470 | | J Virol: |
| | | | | 65(11): 5732-43 |
| 2694-2708 | Linker | Not applicable | Synthetic | Not applicable¹ |
| 2709-3096 | MND | Not applicable | pccl-c-MNDU3c- | Challita et al. |
| | | | x2 | (1995) |
| | | | | J. Virol. 69: 748-755 |
| 3097-3124 | Linker | Not applicable | Synthetic | Not applicable |
| 3125-4783 | I2S, human | Not applicable | Synthetic | Not applicable |
| 4784-4891 | HIV-1 ppt and | Accession | pNL4-3 | Maldarelli, |
| | part of U3 | #M19921.2 | | et. al. (1991) |
| | | nt 9005-9110 | | J Virol: |
| | | | | 65(11): 5732-43 |
| 4892-5008 | HIV-1 part of U3 | Accession | pNL4-3 | Maldarelli, |
| | (399bp deletion) | #M19921.2 | | et. al. (1991) |
| | and R | nt 9511-9627 | | J Virol: |
| | | | | 65(11): 5732-43 |
| 5009-5032 | Synthetic polyA | Not applicable | Synthetic | Levitt, N. Genes |
| | | | | & Dev (1989) |
| | | | | 3: 1019-1025 |
| 5033-5051 | Linker | Not applicable | Synthetic | Not Applicable |
| 5052-7524 | pUC19 backbone | Accession | pUC19 | New England |
| | | #L09137.2 | | Biolabs |
| | | nt 2636-2686 | | (Attachment 1) |

TABLE 2
pEF1α-I2S LVV

| | | GenBank | | |
|---|---|---|---|---|
| Nucleotides | Identity | Reference | Source Name | Citation |
| 1-185 | pUC19 plasmid | Accession | pUC19 | New England |
| | backbone | #L09137.2 | | Biolabs |
| | | nt 1-185 | | (Attachment 1) |
| 185-222 | Linker | Not applicable | Synthetic | Not applicable |
| 223-800 | CMV | Not Applicable | pHCMV | (1994) PNAS 91: |
| | | | | 9564-68 |
| 801-1136 | R, U5, PBS, and | Accession | pNL4-3 | Maldarelli, |
| | packaging | #M19921.2 | | et. al. (1991) |
| | sequences | nt 454-789 | | J Virol: |
| | | | | 65(11): 5732-43 |
| 1137-1139 | Gag start codon | Not Applicable | Synthetic | Not applicable |
| | (ATG) changed | | | |
| | to stop codon | | | |
| | (TAG) | | | |
| 1140-1240 | HIV-1 gag | Accession | pNL4-3 | Maldarelli, |
| | sequence | #M19921.2 | | et. al. (1991) |
| | | nt 793-893 | | J Virol: |
| | | | | 65(11): 5732-43 |
| 1241-1243 | HIV-1 gag | Not Applicable | Synthetic | Not applicable |
| | sequence | | | |
| | changed to a | | | |
| | second stop | | | |
| | codon | | | |
| 1244-1595 | HIV-1 gag | Accession | pNL4-3 | Maldarelli, |
| | sequence | #M19921.2 | | et. al. (1991) |
| | | nt 897-1248 | | J Virol: |
| | | | | 65(11): 5732-43 |
| 1596-1992 | HIV-1 pol | Accession | pNL4-3 | Maldarelli, |
| | cPPT/CTS | #M19921.2 | | et. al. (1991) |
| | | nt 4745-5125 | | J Virol: |
| | | | | 65(11): 5732-43 |
| 1993-2517 | HIV-1, isolate | Accession | PgTAT-CMV | Malim, M. H. |
| | HXB3 env | #M14100.1 | | Nature (1988) |
| | region (RRE) | nt 1875-2399 | | 335: 181-183 |
| 2518-2693 | HIV-1 env | Accession | pNL4-3 | Maldarelli, |
| | sequences S/A | #M19921.2 | | et. al. (1991) |
| | | nt 8290-8470 | | J Virol: |
| | | | | 65(11): 5732-43 |
| 2694-2698 | Linker | Not applicable | Synthetic | Not applicable |
| 2699-3242 | EF1alpha/HTLV | Not applicable | Synthetic | Takebe et al. |
| | promoter | | | (1988) |
| | | | | MCB: 8(1): |
| | | | | 466-472 |
| | | | | Kim DW et al. |
| | | | | (1990), Gene: |
| | | | | 91(2): 217-223 |
| 3243-3258 | Linker | Not applicable | Synthetic | Not applicable |
| 3259-4917 | I2S, human | Not applicable | Synthetic | Not applicable |
| 4918-5025 | HIV-1 ppt and | Accession | pNL4-3 | Maldarelli, |
| | part of U3 | #M19921.2 | | et. al. (1991) |
| | | nt 9005-9110 | | J Virol: |
| | | | | 65(11): 5732-43 |
| 5026-5142 | HIV-1 part of U3 | Accession | pNL4-3 | Maldarelli, |
| | (399bp deletion) | #M19921.2 | | et. al. (1991) |
| | and R | nt 9511-9627 | | J Virol: |
| | | | | 65(11): 5732-43 |
| 5143-5166 | Synthetic polyA | Not applicable | Synthetic | Levitt, N. Genes |
| | | | | & Dev (1989) |
| | | | | 3: 1019-1025 |
| 5167-5185 | Linker | Not applicable | Synthetic | Not Applicable |
| 5186-7658 | pUC19 backbone | Accession | pUC19 | New England |
| | | #L09137.2 | | Biolabs |
| | | nt 2636-2686 | | (Attachment 1) |

### Example 2

### Fibroblasts Transduced with Lentiviral Vectors Encoding I2S

Human fibroblasts deficient in I2S activity because of homozygous mutations in the I2S gene (I2S^{-/-} cells) were cultured in Dulbecco's Modified Eagle Medium (DMEM) plus 10% fetal bovine serum (FBS) for twenty-four hours prior to transduction. Cultured 12S^{-/-} cells were resuspended at 5.0E4 cells/mL of DMEM plus 10% FBS and two mL of this cell suspension were plated per well in a 6-well tissue culture plate and placed at 37° C. Twenty-four hours post cell seeding, cells were transduced with one mL of either unpurified lentiviral vector. One mL of DMEM plus 10% FBS was added to a control well and the cells were placed in a 37° C. incubator. Twenty-four hours post transduction, a complete media exchange was performed. Forty-eight hours post transduction, 250 uL of supernatant from each well was removed to a sterile Eppendorf tube and frozen at −80° C. Cells were washed with one mL phosphate buffered saline and lifted using 0.5 mL of 1× TryplE Express Enzyme (Thermo Fisher). Cells were removed to two sterile Eppendorf tubes per sample and pelleted for five minutes at 1500 rpm. The supernatant was aspirated and cell pellets are frozen at −80° C.

### Example 3

### Protein Expression in Cells Transduced with Lentiviral Vectors Encoding I2S

Frozen cell pellets from wild type control cells, I2S^{-/-} cells, and I2S^{-/-} cells transduced with the lentiviral vectors encoding I2S (pMND-I2S and pEF1α-I2S) are thawed on ice for Western blotting. 300 μL of mammalian protein extraction reagent and 3 μL of 100X HALT protease inhibitor cocktail (ThermoFisher) is added to each cell pellet. Pellets are resuspended by pipetting gently up and down and cells are incubated for 10 minutes at room temperature on a plate rocker. Cells are centrifuged for fifteen minutes at 4° C. at 14,000 rpm and supernatants are removed to sterile Eppendorf tubes. Loading dye is prepared by adding 25 μL β-mercaptoethanol to 4754, 4× Laemmli sample buffer (Bio-Rad). Samples are mixed in a 3:1 sample to loading dye ratio with 30 μL prepared loading dye to 90 μL sample. 20 μL of each sample and 8 μL Precision Plus Protein Kaleidoscope ladder are loaded into the wells of a NuPage 4-12 Bis-Tris protein gel. Gels are run in 1×MES SDS running buffer for 40 minutes at 200V.

Gels are transferred using an iBlot transfer stack on the iBlot 7 minute transfer system. Membranes are rinsed in 1×Tris-buffered saline for five minutes at room temperature. Membranes are incubated in Odyssey blocking buffer plus a 1:500 dilution of rabbit anti-I2S antibody (Abcam ab96498) and a 1:1000 dilution of mouse anti-O-actin antibody (Abcam ab3280) at 4° C. The next morning, membranes are rinsed three times in Tris-buffered saline for five minutes at room temperature. A secondary antibody cocktail containing a 1:1000 dilution of 800RD donkey anti-mouse IgG (Licor 926-32212) and a 1:1000 dilution of 680RD donkey anti-rabbit IgG (Licor 926-68073) in Odyssey blocking buffer. Membranes are incubated for one hour at room temperature in secondary antibody cocktail and rinsed three times with Tris-buffered saline for five minutes at room temperature. Blots are imaged on a Licor Odyssey CLX imaging system.

A Western blot of I2S expression in wild type control cells, I2S^{-/-} cells, and 12S^{-/-} cells transduced with the lentiviral vectors encoding I2S (pMND-I2S and pEF1α-I2S) will be performed.

### Example 4

### Restoration of I2S Activity in I2S^{-/-} Cells Transduced with Lentiviral Vectors Encoding I2S

Cell pellets from wild type control cells, I2S−/− cells (GM01929, GM13203), and I2S−/− cells transduced with the lentiviral vectors encoding I2S (pMND-I2S and pEF1α-I2S) were resuspended in 150 μL PBS buffer containing 20 μl of lead acetate buffer, containing a 4-methylumbellieryl (4-MU) fluorescent reporter (0.1 M sodium acetate (NaAc), 0.1M acetic acid, 10 mM plumbic acetate, 25 mM 4-MU-α-2-sulfate, pH 5.0). Fluorometric measurement of IDS activity was calculated based on cleavage of the 4-MU-α-2-sulfate substrate (see Civallero et al. Clinica Chimica Acta 372 (2006) 98-102). 15 to 25 μg total protein of cell lysate or cell supernatant in the same PBS/acetate buffer were incubated at 37° C. After 24 hours, 40 μl of McIlvaine buffer (0.2M citric acid, 0.4M NaPO4, 0.02% sodium azide, pH 4.5) was added, and the reaction was incubated for an additional 24 hours at 37° C. For supernatants, 15 to 25 total protein was treated as above. Fluorescence was measured using a Molecular Devices SpectraMax M2 spectrofluorimeter.

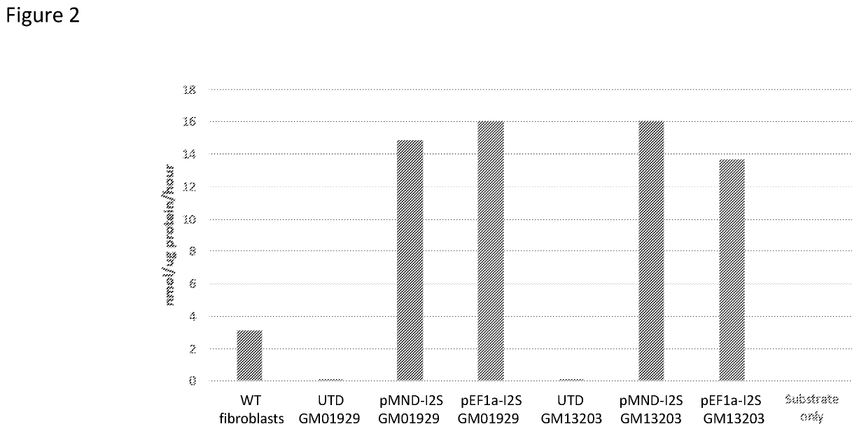

The results of the enzymatic assay corroborate the protein overexpression in transduced patient fibroblasts compared to wild type (WT) fibroblasts. I2S activity in patient cells was restored by transduction with both lentiviral vectors (pMND-125 and pEF1α-I2S). Patient cells transduced with either vector showed IDS activity three to four fold greater than activity in wild type cells. FIG. 2.

### Example 5

### Active Enzymatic Expression in hCD34⁺ Cells Transduced with LVV Encoding I2S

Human CD34+ cells were transduced with a lentiviral vector (LVV) comprising an MND or EF1α promoter linked to a polynucleotide encoding I2S (MPS II). Cells were prestimulated in cytokine containing media for 48 hours and transduced for 24 hours at an MOI of either 5, 15 or 30 using 200 μg/mL poloxamer 338 and 10 μM PGE₂. After transduction, cells were plated in methylcellulose and cultured for 12 days to allow for hematopoietic progenitor colony formation or cultured in cytokine containing media for 7 days. Samples were analyzed for cell growth, VCN, individual colony VCN and % LVV+ cells, and I2S activity in pellets and supernatant.

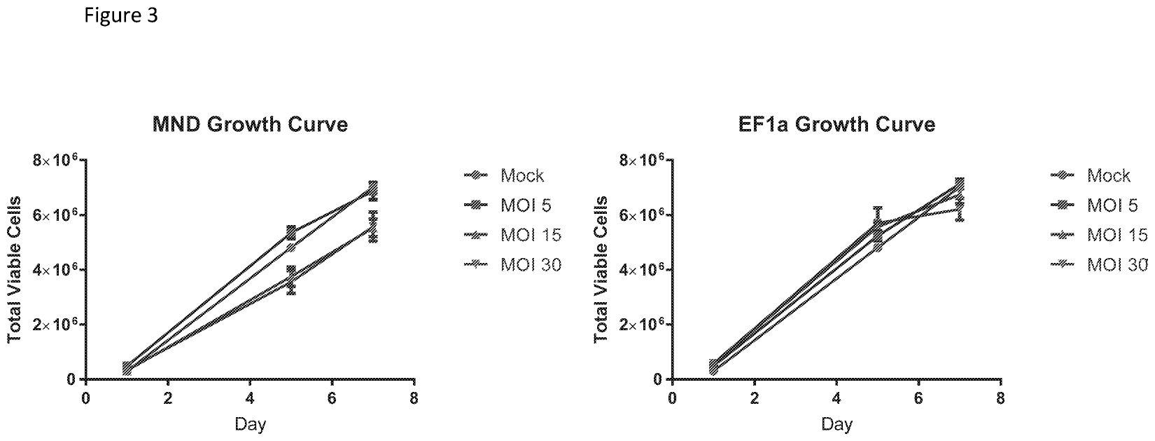

Cells in culture exhibited similar growth kinetics compared to mocks, indicating that neither LVV resulted in toxicity. FIG. 3.

VCN was measured in transduced cells cultured in cytokines for 7 days or 14 days.

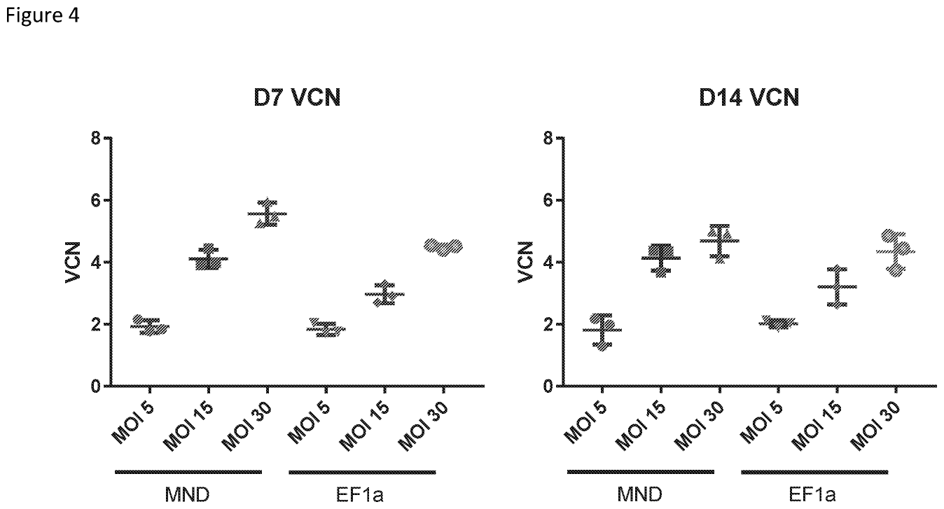

Transduction with each vector resulted in high VCN across all MOIs. The MND-containing vector reached higher VCNs than the EF1α-containing vector. FIG. 4.

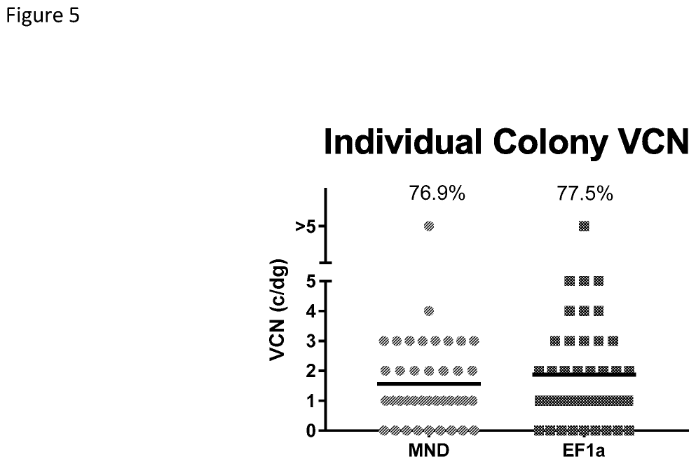

Individual colonies from MOI 5 samples were plucked from day 12 methylcellulose cultures and analyzed by qPCR for VCN and % LVV+ cells. Both vectors resulted in an average VCN above 1.5 and high % LVV+. The EF1α vector transduced the cells slightly more efficiently. FIG. 5.

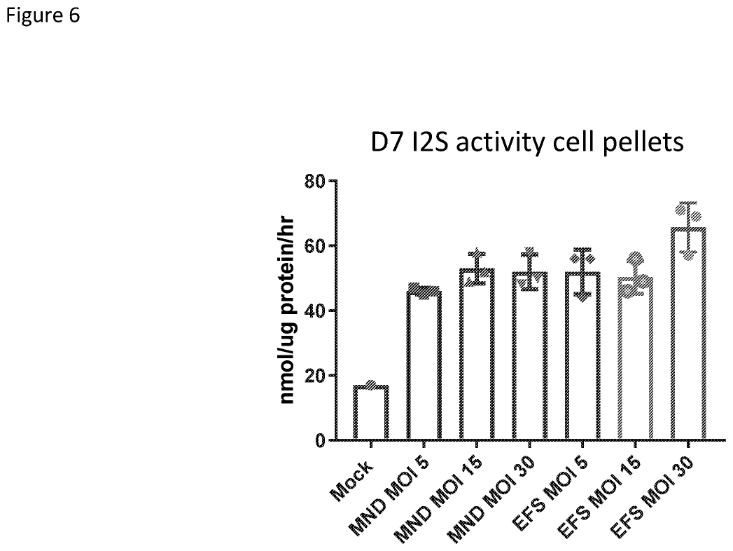

After transduced cells were cultured in cytokines for 7 days, cell pellets were assayed for I2S activity. I2S activity was nearly equivalent in cell pellets across MOIs for both vectors. FIG. 6.

### Example 6

### In Vivo I2S Gene Therapy Model

Mice with I2S mutations will be administered HSCs transduced with lentiviral vectors encoding I2S and phenotypically characterized. I2S mutant mice will undergo treatment to ablate bone marrow hematopoietic stem cells and administered HSCs transduced with lentiviral vectors encoding I2S at no more than 2 weeks of age.

Clinical assessment will be performed beginning the first day after initial treatment, and, at ˜4 weeks of age, mice will undergo clinical assessment, which includes observation for tremors, general body condition, weight gain (weekly, starting at ˜4 weeks of age), grip strength (biweekly, beginning at ˜8 weeks of age), rotarod (at ˜13, 18 weeks of age), and gait analysis (at ˜16 and ˜24 weeks of age).

In addition to the behavioral assays, mice will be tested post-transplant for other parameters to assess their general health and immune system reconstitution after hematopoietic stem cell therapy including full clinical blood chemistry panels, CNS gross morphology and histological analysis to assess storage material, neuronal and glial cell numbers, and morphology (e.g., axonal degeneration) in sagittal sections (to capture multiple brain regions in each section), evidence of cross-correction (expression) in tissues affected by I2S deficiency, I2S enzyme activity in blood/brain/tissue lysates, bone marrow morphology, measurement of vector copy number in mouse bone marrow at the end of all experiments; and identification of engrafted cells.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A polynucleotide comprising:
(a) a left (5′) lentiviral LTR;
(b) a Psi (ψ) packaging signal;
(c) a retroviral export element;
(d) a central polypurine tract/DNA flap (cPPT/FLAP);
(e) a promoter operably linked to a polynucleotide encoding iduronate 2-sulfatase (I2S) polypeptide; and
(f) a right (3′) lentiviral LTR.

2. The polynucleotide of claim 1, wherein the lentivirus is selected from the group consisting of: HIV (human immunodeficiency virus; including HIV type 1, and HIV type 2); visna-maedi virus (VMV) virus; caprine arthritis-encephalitis virus (CAEV); equine infectious anemia virus (EIAV); feline immunodeficiency virus (Hy); bovine immune deficiency virus (BIV); and simian immunodeficiency virus (SIV).

3. The polynucleotide of claim 1 or claim 2, wherein the lentivirus is HIV-1 or HIV-2.

4. The polynucleotide of any one of claims 1-3, wherein the lentivirus is HIV-1.

5. The polynucleotide of any one of claims 1-4, wherein the promoter of the 5′ LTR is replaced with a heterologous promoter selected from the group consisting of: a cytomegalovirus (CMV) promoter, a Rous Sarcoma Virus (RSV) promoter, and a Simian Virus 40 (SV40) promoter.

6. The polynucleotide of any one of claims 1-5, wherein the 3′ LTR comprises one or more modifications.

7. The polynucleotide of any one of claims 1-6, wherein the 3′ LTR comprises one or more deletions that prevent viral transcription beyond the first round of viral replication.

8. The polynucleotide of any one of claims 1-6, wherein the 3′ LTR comprises a deletion of the TATA box and Sp1 and NF-κB transcription factor binding sites in the U3 region of the 3′ LTR.

9. The polynucleotide of any one of claims 1-6, wherein the 3′ LTR is a self-inactivating (SIN) LTR.

10. The polynucleotide of any one of claims 1-9, wherein the promoter operably linked to a polynucleotide encoding an I2S polypeptide is selected from the group consisting of: an integrin subunit alpha M (ITGAM; CD11b) promoter, a CD68 promoter, a C-X3-C motif chemokine receptor 1 (CX3CR1) promoter, an ionized calcium binding adaptor molecule 1 (IBA1) promoter, a transmembrane protein 119 (TMEM119) promoter, a spalt like transcription factor 1 (SALL1) promoter, an adhesion G protein-coupled receptor E1 (F4/80) promoter, a myeloproliferative sarcoma virus enhancer, negative control region deleted, d1587rev primer-binding site substituted (MND) promoter and transcriptionally active fragments thereof.

11. The polynucleotide of any one of claims 1-9, wherein the promoter operably linked to a polynucleotide encoding an I2S polypeptide comprises an elongation factor 1 alpha (EF1a) promoter or transcriptionally active fragment thereof.

12. The polynucleotide of any one of claims 1-9, wherein the promoter operably linked to a polynucleotide encoding an I2S polypeptide is a short EF1α promoter.

13. The polynucleotide of any one of claims 1-9, wherein the promoter operably linked to a polynucleotide encoding an I2S polypeptide is a long EF1a promoter.

14. The polynucleotide of any one of claims 1-13, wherein the polynucleotide encoding the I2S polypeptide is a cDNA.

15. The polynucleotide of any one of claims 1-14, wherein the polynucleotide encoding the I2S polypeptide is codon optimized for expression.

16. A polynucleotide comprising:
(a) a left (5′) HIV-1 LTR;
(b) a Psi (ψ) packaging signal;
(c) an RRE retroviral export element;
(d) a cPPT/FLAP;
(e) an MND promoter or EF1α promoter operably linked to a polynucleotide encoding an I2S polypeptide; and
(f) a right (3′) HIV-1 LTR.

17. A polynucleotide comprising:
(a) a left (5′) CMV promoter/HIV-1 chimeric LTR;
(b) a Psi (ψ) packaging signal;
(c) an RRE retroviral export element;
(d) a cPPT/FLAP;
(e) an MND promoter or EF1a promoter operably linked to a polynucleotide encoding an I2S polypeptide; and
(f) a right (3′) SIN HIV-1 LTR.

18. The polynucleotide of any one of claims 1-17, further comprising a bovine growth hormone polyadenylation signal or a rabbit β-globin polyadenylation signal.

19. A mammalian cell transduced with a lentiviral vector comprising a polynucleotide according to any one of claims 1-18.

20. The mammalian cell of claim 19, wherein the cell is a hematopoietic cell.

21. The mammalian cell of claim 19 or claim 20, wherein the cell is a CD34⁺ cell.

22. The mammalian cell of any one of claims 19-21, wherein the cell is a stem cell or progenitor cell.

23. A producer cell comprising: a first polynucleotide encoding gag, a second polynucleotide encoding pol, a third polynucleotide encoding env, and a polynucleotide according to any one of claims 1-18.

24. A lentiviral vector produced by the producer cell of claim 23.

25. A composition comprising a lentiviral vector comprising a polynucleotide according to any one of claims 1-18 or a mammalian cell according to any one of claims 19-22.

26. A pharmaceutical composition comprising a pharmaceutically acceptable carrier and a lentiviral vector comprising a polynucleotide according to any one of claims 1-18 or a mammalian cell according to any one of claims 19-22.

27. A method of treating Hunter Syndrome, comprising administering to a subject a lentiviral vector comprising a polynucleotide according to any one of claims 1-18; a cell transduced with a lentiviral vector comprising a polynucleotide according to any one of claims 1-18; or a mammalian cell according to any one of claims 19-22.

28. A method of treating Hunter Syndrome, comprising administering to a subject a pharmaceutical composition of claim 26.

29. A method of decreasing at least one symptom associated with Hunter Syndrome in a subject comprising administering to a subject a lentiviral vector comprising a polynucleotide according to any one of claims 1-18; a cell transduced with a lentiviral vector comprising a polynucleotide according to any one of claims 1-18; or a mammalian cell according to any one of claims 19-22.

30. A method of decreasing at least one symptom associated with Hunter Syndrome in a subject comprising administering to a subject a pharmaceutical composition of claim 26.

31. The method of claim 29 or claim 30, wherein the at least one symptom is selected from the group consisting of: build up of GAGs, thickening of organ and tissues, difficulty breathing, difficulty swallowing, joint stiffness, cognitive function decline, and motor function decline.
